(19) United States
(12) Patent Application Publication
(10) Pub. No.: US 2024/0058478 A1
(43) Pub. Date: Feb. 22, 2024

(54) FOLATE RECEPTOR-TARGETED CONJUGATES, COMPOSITIONS, AND DELIVERY TO THE CENTRAL NERVOUS SYSTEM

(71) Applicant: PURDUE RESEARCH FOUNDATION, West Lafayette, IN (US)

(72) Inventors: Spencer S. GARDEEN, Bloomington, MN (US); Seth HERR, Indianapolis, IN (US); Philip LOW, West Lafayette, IN (US); Riyi SHI, West Lafayette, IN (US)

(21) Appl. No.: 18/260,598

(22) PCT Filed: Jan. 6, 2022
(86) PCT No.: PCT/US2022/011392

§ 371 Date: Jul. 6, 2023

Related U.S. Application Data
(60) Provisional application No. 63/134,860, filed on Jan. 7, 2021.

(51) Int. Cl. A61K 49/00; A61K 47/55; A61K 31/095; A61P 25/00

(52) U.S. Cl. CPC A61K 49/0032; A61K 47/551; A61K 49/0052; A61K 31/095; A61P 25/00

(57) ABSTRACT

A conjugate comprising a ligand that targets a folate receptor linked to an acrolein scavenger drug; a composition comprising same; a method of using the conjugate or the composition to scavenge acrolein, alone or in further combination with other reactive aldehyde species, in a subject with neurotrauma; and a method of delivering an acrolein scavenger or an imaging agent to the central nervous system (CNS) of a patient with inflammation in the CNS.

### CROSS REFERENCE TO RELATED APPLICATIONS

This application claims priority to U.S. provisional patent application No. 63/134,860, which was filed Jan. 7, 2021, and which is hereby incorporated by reference in its entirety.

### TECHNICAL FIELD

This disclosure relates to folate receptor-targeted conjugates, compositions comprising same, and methods of use to deliver a payload, such as an imaging agent or an anti-inflammatory agent, to the central nervous system.

### BACKGROUND

The central nervous system (CNS) comprises the brain, the spinal cord, and the eyes. The CNS is bathed by a clear, plasma-like fluid referred to as cerebrospinal fluid (CSF).

There are several ways vitamin folate enters the CNS. Folate derivatives, such as 5-methyltetrahydrofolate (5-MTHF), also readily enter the CNS. Entry is mediated by folate receptors, primarily folate receptor α (FRα), which is expressed on choroid plexus cells on the basolateral side. These cells co-transport FRα and folate from the blood to the CSF through transcytosis and release of an exosome into the CSF. The exosomes with FRα are collectively referred to as the folate receptor exosome system (FRES). The FRES perfuses the CNS and eventually enters the brain parenchyma, neurons, and glia.

Alternatively, folate can enter the CNS by breakdown of the blood-brain-barrier (BBB), which occurs in many diseases, or by retinal pigment epithelial (RPE) cells, which are in the eye. Entry of FRES into the optic nerve, or into the eye through the optic nerve head, is also possible.

Folate receptor β (FRβ) and FRα are expressed on activated macrophages and microglia. Activated macrophages can be present at a site of inflammation in the CNS, such as inflammation at a site of spinal trauma (e.g., injury) (see, e.g., Xia et al., Blood 113(2): 438-446 (2009); and Fujiwara et al., Inflammation and Allergy 4(3): 281-286 (2005)). These cells facilitate uptake of FRES into CNS tissues.

Production of oxidative stress and free radicals has been implicated as a key contributor to CNS inflammation. Specifically, α/β unsaturated aldehydes (e.g., acrolein, methylenedioxyamphetamine, and 4-hydroxynonenal) are toxic and reactive compounds, which damage CNS cells and generate free radicals. These reactive aldehydes perpetuate oxidative stress and inflammation, which enhance cellular degeneration and functional loss in multiple CNS pathologies. In neurotrauma, the accumulation of neurotoxic aldehydes at significantly elevated levels is detrimental to healthy recovery.

In view of the above, it is desirable to target delivery of an agent, which can counter the effects of free radicals and oxidative stress, to the CNS. It is an object of the present disclosure to provide conjugates, compositions, and methods for targeted delivery of an acrolein scavenger to the CNS to counter the effects of free radicals and oxidative stress in an effort to reduce inflammation and to treat neurotrauma. It is another object of the present disclosure to provide methods for imaging inflammation, such as that associated with neurotrauma, in the CNS of a patient. The imaging methods can be used to diagnose disease, such as the progression thereof (e.g., as evidenced by the existence and extent of inflammation), and injury (e.g., neurotrauma), such as the extent thereof, to the CNS as well as to assess efficacy of treatment (which, in certain applications, can include prophylaxis). These and other objects and advantages, as well as additional inventive features, will become apparent from the detailed description provided herein.

### SUMMARY

Provided is a method of delivering a payload to a patient with inflammation in the central nervous system (CNS). The method comprises administering to the patient with inflammation in the CNS an effective amount of (i) a conjugate comprising (a) a ligand, which binds folate receptor α (FRα) and/or folate receptor β (FRβ), (b) a payload selected from an acrolein scavenger and an imaging agent, and (c) a linker, which links the ligand and the payload, or (ii) a pharmaceutical composition comprising the conjugate and a pharmaceutically acceptable carrier. The ligand can be a folate or a derivative or analog thereof. The ligand can be 5-methyltetrahydrofolate (5-MTHF). The linker can be hydrophilic. The linker can be self-immolative. The linker can comprise a hydrophilic amino acid, a polyethylene glycol polymer, a peptidoglycan, a sugar, or a combination of two or more of the preceding. The linker can comprise a hydrophilic amino acid, a polyethylene glycol polymer, or a combination thereof. The imaging agent can be a near infrared (NIR) dye. The NIR dye can be S0456. The conjugate can be OTL38. The imaging agent can be a magnetic resonance imaging (MRI) contrast agent. The MRI contrast agent can be gadolinium. The imaging agent can be a positron emission tomography/computed tomography (PET/CT) contrast agent or a single photon emission computed tomography (SPECT). The PET/CT contrast agent can be technetium. When the payload is an imaging agent, the method can further comprise imaging the patient. The payload can be transported to an eye. The acrolein scavenger can be hydralazine. The acrolein scavenger can be dimercaprol, in which each thiol group is linked to a linker, which is linked to a ligand. Each thiol group in dimercaprol can be linked to a linker comprising a penicillamine and the linker can be attached to the ligand by the penicillamine.

The patient can have neurotrauma, stroke, epilepsy, multiple sclerosis, motor neuron disease, a movement disorder (e.g., Parkinson's disease), Alzheimer's disease, amyotrophic lateral sclerosis, Creutzfeldt-Jakob disease, depression, anxiety, schizophrenia, Guillain-Barre syndrome, an infection or inflammatory disease of the central nervous system, optic atrophy, retinal degeneration, ocular proptosis, ocular trauma, an ocular inflammatory disorder, glaucoma, or macular degeneration.

Further provided is a conjugate comprising (a) a ligand, which binds FRα and/or FRβ, (b) a linker, and (c) an acrolein scavenger, wherein the linker links the ligand to the acrolein scavenger. The ligand can be a folate or a derivative or an analog thereof. The ligand can be 5-MTHF. The acrolein-scavenging drug can be hydralazine. The acrolein scavenger can be dimercaprol, in which each thiol group is linked to a linker, which is linked to a ligand. Each thiol group in dimercaprol can be linked to a linker comprising a penicillamine and the linker can be attached to the ligand by the penicillamine. The linker can be hydrophilic. The linker can be self-immolative. The linker can comprise a hydrophilic amino acid, a polyethylene glycol polymer, a peptidoglycan, a sugar, or a combination of two or more of the preceding. The linker can comprise a hydrophilic amino acid, a polyethylene glycol polymer, or a combination thereof.

In view of the above, also provided is a pharmaceutical composition. The pharmaceutical composition comprises a conjugate comprising (a) a ligand, which binds FRα and/or FRβ, (b) a linker, and (c) an acrolein scavenger, wherein the linker links the ligand to the acrolein scavenger, and a pharmaceutically acceptable carrier.

Still further provided is a method of treating a patient for neurotrauma, such as spinal trauma (e.g., spinal injury) or brain trauma (e.g., brain injury). The method comprises administering to the patient a therapeutically effective amount of (i) a conjugate comprising (a) a ligand, which binds FRα and/or FRβ, (b) a linker, and (c) an acrolein scavenger, wherein the linker links the ligand to the acrolein scavenger, or (ii) a pharmaceutical composition comprising the conjugate and a pharmaceutically acceptable carrier.

### FIGURES

FIG. 1 shows the structure of a folate-hydralazine conjugate.

FIG. 2 shows the reaction of the activation of hydralazine for linkage to folate.

FIG. 3 shows the structure of a folate-linker.

FIG. 4 shows the reaction of a folate-linker with activated hydralazine.

FIG. 5A is a graph of control (CTL; n=3), spinal injury (INJURY; n=4), spinal injury treated with hydralazine (INJ+HDZ; n=4), and spinal injury treated with a conjugate comprising folate linked to hydralazine (INJ+HDZ-FOL; n=4) vs. blood pressure (BP) normalized to baseline for systolic and diastolic blood pressure measured on day 4.

FIG. 5B is a graph of control (CTL; n=3), spinal injury (INJURY; n=4), spinal injury treated with hydralazine (INJ+HDZ; n=4), and spinal injury treated with a conjugate comprising folate linked to hydralazine (INJ+HDZ-FOL; n=4) vs. blood pressure (BP) normalized to baseline for systolic and diastolic blood pressure measured on day 7.

FIG. 6A shows images from rat spinal cords on day 8 post-spinal injury. HDZ=hydralazine. F-HDZ=folate-hydralazine.

FIG. 6B is a graph of control, injury (INJ), spinal injury treated with hydralazine (INJ+HDZ), and spinal injury treated with a conjugate comprising folate linked to hydralazine (INJ+HDZ-FOL) vs. acrolein levels detected by immunohistochemistry and expressed as mean pixel intensity. ns=not significant. *=P<0.10. **=P<0.01. ***=P<0.001.

FIG. 7A is a chromatogram of activated hydralazine.

FIG. 7B is a chromatogram of folate linked to hydralazine.

FIG. 8 shows images of brains divided in half with a single sagittal cut through the corpus collosum, revealing the choroid plexus in the left and right hemispheres. Corresponding ventral side of the spinal cords are shown to the left of each brain. Red autofluorescence reveals the tissue structure. Green fluorescence indicates OTL38 signal. The left column (OTL38, n=5) reveals strong signal in the choroid plexus. In the middle column (OTL38+Fol-Glu, n=4) minimal uptake is observed. In the far-right corner, Control (non-injected, n=1), no signal is observed. FIG. 9 is a graph of mean fluorescent intensity vs. time for CSF and blood. n=1-4 rats/timepoint.

FIG. 10 shows ex vivo imaging of spinal cords of rats with a moderate grade spinal cord contusion injury (SCI) and injected with OTL38 or simultaneously with OTL38 and Fol-Glu on day seven after injury.

FIGS. 11A-11B show transverse spinal cord sections of the rostral portions (FIG. 11A) and epicenters (FIG. 11B) of a rat with SCI and injected seven days after injury with OTL38.

FIG. 11C shows transverse spinal cord sections of the epicenters of a rat with SCI and injected seven days after injury with the unconjugated near infrared dye (NIR) S0456.

FIG. 11D is a bar graph of mean fluorescence intensity vs. rostral portion and epicenter showing OTL38 uptake in for the regions imaged in FIGS. 11A-11B (students t-test, **p<0.01).

FIG. 12 shows transverse sections of SCI rat epicenter regions stained and imaged using 10× immunofluorescent microscopy with anti-IBA1 staining shown in green and anti-FRβ staining shown in red. The merge shows instances of co-localization, with one instance encircled in yellow.

FIGS. 13A-13B show a precise surface laceration injury in the cortex of the left hemisphere of the brain. FIG. 13A is a cartoon indicating the area of injury (blue circle) and the area imaged (blue rectangle). FIG. 13B is an image showing enhanced signal around the injury area (blue circle). Increased signal around the choroid plexus is indicated with the blue arrow. The right hemisphere cortex served as an internal control (uninjured, no signal).

FIG. 14 shows images of the brain and spinal cord of a rat with a controlled cortical impact (CCI) injury and injected with OTL38. Increased signal around the choroid plexus and FRES entry to the CNS/CSF is indicated with the blue arrow. The red arrow shows positive targeting of the 6 mm circular injury. The yellow arrow points to signal on the dorsal surface of the spinal cord.

FIG. 15A shows MRI at various sections of a rat with a CCI injury and injected with fol-Gad. Blue arrows point to brain injury with enhanced contrast, and corresponding transverse section bregmas are shown at the tops of the images in millimeters.

FIG. 15B shows MRI at various sections of a rat with a CCI injury and not injected with fol-Gad. Red arrows point to brain injury with no obvious enhancement in signal, and corresponding transverse section bregmas are shown at the tops of the images in millimeters.

FIG. 16A shows the imaging of the live rat injected with OTL38 24 hours earlier. Red and green colors indicate signal from the dye, with red showing the most intense regions. The blue arrow indicates intense red signal at the eye.

FIG. 16B shows the PET/CT scan of the rat with the closed head injury and injected with Fol-Tc. The purple color indicates signal from Fol-Tc uptake. The blue arrows show intense signal around the retinal areas of the eye. The yellow arrows indicate the choroid plexus.

FIG. 16C is a diagram of where sections from the eye were sliced and analyzed for the images shown in FIGS. 16E-16F.

FIG. 16D shows an image of a rat injected with unconjugated NIR dye.

FIG. 16E shows an image of a slice taken from the retinal area nearest the optic nerve (corresponding to the red box in FIG. 16C) of a rat injected with OTL38.

FIG. 16F shows an image of a slice taken from the area closer to the cornea and corresponding to the black box in FIG. 16C of the same rat injected with OTL38.

FIGS. 17A-17I are images of scans of whole eyes removed from mice at various timepoints (2 hours (FIG. 17B) or 1 (FIG. 17C), 2 (FIG. 17D), 3 (FIG. 17E), 4 (FIG. 17F), 7 (FIG. 17G), 14 (FIG. 17H) or 21 (FIG. 17I) days) after intravenous injection with OTL38 as compared to control mice (FIG. 17A), which did not receive an injection of OTL38.

FIGS. 18A-18E are images of scans of whole eyes removed from mice after intravenous injection with OTL38 alone or with subsequent administration of folate-glucosamine (Fol-Glc). Images of eyes of noninjected mice are shown in FIG. 18A. Images of eyes of mice injected with OTL38 and sacrificed three days later are shown in FIG. 18B, whereas images of eyes of mice injected with OTL38 on day 1, injected with 10 mmol Fol-Glc on day 2, and sacrificed on day 3 are shown in FIG. 18C, images of eyes of mice injected with OTL38 on day 1 and sacrificed on day 4 are shown in FIG. 18D, and images of eyes of mice injected with OTL38 on day 1, injected with 10 mmole Fol-Glc on days 2 and 3, and sacrificed on day 4 are shown in FIG. 18E.

FIG. 18F is a graph of treatment vs. mean fluorescence intensity for the mice shown in FIGS. 18A-18E.

FIG. 19 shows the images of eyes extracted from treated mice compared to images of eyes extracted from nontreated mice.

FIG. 20 shows the images of eyes extracted from treated mice (Folate-Cy5) compared to images of eyes extracted from nontreated mice. Layers of the eye are labeled: GCL=ganglion cell layer; IPL=inner plexiform layer; INL=inner nuclear layer; OPL=outer plexiform layer; ONL=outer nuclear layer; IS/OS=inner and outer segments of the photoreceptor layer; RPE=retinal pigmented epithelium.

FIGS. 21A-21D show the reaction scheme for folate (2)-dimercaprol.

### DETAILED DESCRIPTION

Provided is a method of delivering a payload to a patient with inflammation in the central nervous system (CNS). The patient can have inflammation due to neurotrauma (e.g., spinal trauma (e.g., spinal injury) or brain trauma (e.g., brain injury)) or inflammation accompanying the onset or existence of a disease or other condition, such as stroke, epilepsy, multiple sclerosis, motor neuron disease, a movement disorder (e.g., Parkinson's disease), Alzheimer's disease, amyotrophic lateral sclerosis (ALS), Creutzfeldt-Jakob disease (CJD), depression, anxiety, schizophrenia, Guillain-Barre syndrome, infection (e.g., viral, bacterial, fungal, or parasitic) or inflammatory disease of the CNS, optic atrophy, retinal degeneration, ocular proptosis, ocular trauma, ocular inflammatory disorders, glaucoma, or macular degeneration (see, e.g., Lucas et al., Br J Pharmacol 147 (Suppl 1): S232-S240 (January 2006)). Such a patient can be identified through physical examination, laboratory analysis that reveals, for example, an elevated level of one or more inflammatory mediators, such as proinflammatory cytokines (e.g., interleukin-1 (IL-1), interleukin-6 (IL-6), interleukin-12 (IL-12), and tumor necrosis factor-α (TNF-α)), elevated levels of immune cells in the cerebrospinal fluid (CSF), or the presence of an infectious agent, for example, and/or imaging analysis that reveals structural changes in the CNS or the presence of inflammatory cells identified by binding of a conjugate comprising an imaging agent as described herein.

Acrolein and other reactive aldehyde species and free radicals contribute to and exacerbate inflammation. In doing so, such compounds can contribute to the adverse effects of disease states, such as those indicated above, and can directly damage cells of the CNS. Scavengers of acrolein and other reactive aldehyde species and free radicals, such as scavengers that are targeted to folate receptors as disclosed herein, can impede the effects of the scavenged compounds on inflammation, thereby reducing the adverse effects of inflammation on various disease states.

The method comprises administering to the patient an effective amount of (i) a conjugate comprising (a) a ligand, which binds folate receptor α (FRα) and/or folate receptor β (FRβ), (b) a payload selected from an acrolein scavenger and an imaging agent, and (c) a linker, which links the ligand and the payload or (ii) a pharmaceutical composition comprising the conjugate and a pharmaceutically acceptable carrier. When the payload is an imaging agent, the method can further comprise imaging the patient. The payload can be transported to an eye.

The method can enable a payload, such as an acrolein scavenger or an imaging agent, which is otherwise unable to reach the CNS, to reach the CNS. The method also can enable a payload, which is otherwise unable to reach the CNS in an effective concentration, to reach the CNS in an effective concentration. The method also can enable a payload, which is able to reach the CNS but is cleared rapidly, to reach the CNS and remain longer.

Still further provided is a method of treating a patient for neurotrauma (e.g., spinal trauma (e.g., spinal injury) or brain trauma (e.g., brain injury)). A nonlimiting example of spinal injury is spinal cord contusion injury (SCI). Examples of brain injury include, but are not limited to, laceration, impaction, and contusion. Inflammation, an increase in acrolein, an increase in α-synuclein aggregation (e.g., due to increased acrolein), an increase in immune cells, and/or a decrease in aldehyde dehydrogenase 2 (ALDH2) (e.g., due to increased acrolein) can present with neurotrauma. With spinal cord injury, such as SCI, acrolein can peak around 48 hours after injury and remain significantly elevated for at least two weeks post-injury (Luo et al., Neurochem Int 44(7): 475-486 (2004)).

The method comprises administering to the patient a therapeutically effective amount of (i) a conjugate comprising (a) a ligand, which binds FRα and/or FRβ, (b) a linker, and (c) an acrolein scavenger, wherein the linker links the ligand to the acrolein-scavenging drug, or (ii) a pharmaceutical composition comprising the conjugate and a pharmaceutically acceptable carrier.

Any suitable route of administration can be used as known in the art. Examples of suitable routes of administration include intravenous, intrathecal, epidural, intracerebroventricular, and intracranial. Intravenous administration is less invasive and, consequently, can be preferred.

An effective amount of the conjugate or compound or the pharmaceutical composition can be determined in accordance with dosage range-finding techniques commonly employed in the art. Methods of the extrapolation of effective dosages in mice and other animals to human subjects are known in the art. Indeed, the dosage of the conjugate (or pharmaceutical composition comprising the conjugate) can vary significantly depending on the condition of the patient, the neurotrauma or the condition causing the inflammation, how advanced the pathology is, the route of administration of the conjugate/composition and CNS distribution, and the possibility of co-usage of other therapeutic treatments (such as additional drugs in combination therapies). The amount of the conjugate/composition required for use will vary not only with the particular application, but also with the characteristics of the subject (such as, for example, age, condition, sex, the subject's body surface area and/or mass, tolerance to drugs) and will ultimately be at the discretion of the attendant physician, clinician, or otherwise. Therapeutically (or prophylactically) effective or diagnostically effective amounts or doses can range, for example, from about 0.05 mg/kg of patient body weight to about 30.0 mg/kg of patient body weight, or from about 0.01 mg/kg of patient body weight to about 5.0 mg/kg of patient body weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of patient body weight. The total amount can be administered in a single or divided dose and can, at the physician's discretion, fall outside of the typical range given herein.

Alternatively, the conjugate (or composition) can be administered in a therapeutically (or prophylactically) or diagnostically effective amount of from about 0.5 g/m to about 500 mg/m², from about 0.5 g/m² to about 300 mg/m², or from about 100 g/m² to about 200 mg/m². In other embodiments, the amounts can be from about 0.5 mg/m² to about 500 mg/m², from about 0.5 mg/m² to about 300 mg/m², from about 0.5 mg/m² to about 200 mg/m², from about 0.5 mg/m² to about 100 mg/m², from about 0.5 mg/m² to about 50 mg/m², from about 0.5 mg/m² to about 600 mg/m², from about 0.5 mg/m² to about 6.0 mg/m², from about 0.5 mg/m² to about 4.0 mg/m², or from about 0.5 mg/m² to about 2.0 mg/m². The total amount can be administered in a single or divided dose and can, at the physician's discretion, fall outside of the typical range given herein. These amounts are based on m² of body surface area.

The conjugate, or a composition comprising the conjugate, can be used to improve the outcome of neurotrauma (e.g., spinal trauma (e.g., spinal injury) or brain trauma (e.g., brain injury)). When the payload is hydralazine, the conjugate, or a composition comprising the conjugate, can be administered to a subject without adversely affecting blood pressure. The conjugate, or a composition comprising the conjugate, also can be used to target, e.g., to treat, inflammation in the CNS, such as inflammation associated with stroke, epilepsy, multiple sclerosis, motor neuron disease, a movement disorder (e.g., Parkinson's disease), Alzheimer's disease, depression, amyotrophic lateral sclerosis, Creutzfeldt-Jakob disease, depression, anxiety, schizophrenia, Guillain-Barre syndrome, an infection (e.g., viral, bacterial, fungal, or parasitic) or inflammatory disease of the central nervous system, optic atrophy, retinal degeneration, ocular proptosis, ocular trauma, an ocular inflammatory disorder, glaucoma, or macular degeneration. The removal of acrolein, alone or in further combination with other reactive aldehyde species, can promote tissue survival and regeneration, reduce neuropathic pain, reduce inflammation, and improve functional motor recovery (Chen et al., J Neurochem 138(2): 328-338 (2016); Park et al., J Neurochem 135(5): 987-997 (2015); Shi et al., Mol Nutr Food Res 55(9): 1320-1331 (2011); Hamann et al., J Neurochem 104(3): 708-718 (2008); Liu-Snyder et al., J Neurosci Res 84(1): 219-227 (2006); Park et al., J Neurochem 129(2): 339-349 (2014); and Tian et al., J Neurochem 141(5): 708-720 (2017)).

The acrolein scavenger can effectively scavenge acrolein and may, and even can, also scavenge other reactive aldehyde species. The acrolein scavenger can be any suitable acrolein scavenger. An example of such a drug is hydralazine, which is approved for the treatment of blood pressure by the Food and Drug Administration (FDA). The acrolein scavenger can be dimercaprol, in which each thiol group is linked to a linker, which is linked to a ligand. Each thiol group in dimercaprol can be linked to a linker comprising a penicillamine and the linker can be attached to the ligand by the penicillamine. Other examples of acrolein scavengers include, but are not limited to, those that contain sulfur (thiol) or nitrogen (amino) or are polyphenolic. Specific examples of acrolein-scavenging drugs include, for example, γ-aminobutyric acid (GABA), N-acetylcysteine (NAC), cysteamine, N-benzylhydroxylamine, apresoline, 2-hydrozino-4,6-dimethylpyrimidine (2-HDP), and phenelzine. For a review on acrolein scavengers, see, e.g., Zhu et al., Mol Nutr Food Res 55(9): 1375-1390 (September 2011)).

In view of the above, also provided is a conjugate comprising a ligand, which binds FRα and/or FRβ, a linker, and an acrolein scavenger, wherein the linker links the ligand to the acrolein scavenger. The folate receptor, such as FRβ, can be on the surface of a cell, such as a cell in the CSF, such as an activated macrophage or other immune cell at a site of inflammation.

The imaging agent can be any suitable imaging agent. The imaging agent can be a near infrared (NIR) dye. The NIR dye can be S0456 or cyanine 5 (Cy5). The conjugate can be OTL38. Other imaging agents include, but are not limited to, fluorescein, fluorescein isothiocyanate (FITC), rhodamine, LS288, heptamethine cyanine dye (HMCD), SS180, acridine orange (AO), IRDye800CW, IR783, IR825, ZW800-1, phycoerythrin, and indocyanine (e.g., indocyanine green). The imaging agent can be a radioactive imaging agent, such as ¹⁸F, ⁴⁴Sc, ⁴⁷Sc, ⁵²Mn, ⁵⁵Co, ⁶⁴Cu, ⁶⁷Cu, ⁶⁷Ga, ⁶⁸Ga, ⁸⁶Y, ⁸⁹Zr, ⁹⁰Y, ^{99m}Tc, ¹¹¹In, ^{114m}In, ^{117m}Sn, ¹²⁴I, ¹²⁵I, ¹³¹I, ¹⁴⁹Tb, ¹⁵³Sm, ¹⁵²Tb, ¹⁵⁵Tb, ¹⁶¹Tb, ¹⁷⁷Lu, ¹⁸⁶Re, ¹⁸⁸Re, ²¹²Pb, ²¹²Bi, ²¹³Bi, ²²³Ra, ²²⁴Ra, ²²⁵Ab, ²²⁵Ac, or ²²⁷Th. The imaging agent can be a magnetic resonance imaging (MRI) contrast agent. The MRI contrast agent can be gadolinium. The imaging agent can be a positron emission tomography/computed tomography (PET/CT) contrast agent or a single photon emission computed tomography (SPECT). The PET/CT contrast agent can be technetium. The radioactive isotope can be chelated with a chelator such as DOTA (1,4,7,10-tetraazacyclododecane-1,4,7,10-tetraacetic acid) or a derivative thereof;

TETA (1,4,8,11-tetraazacyclotetradecane-1,4,8,11-tetraacetic acid) or a derivative thereof; HEHA (1,2,7,10,13-hexaazacyclooctadecane-1,4,7,10,13,16-hexaacetic acid) or a derivative thereof; PEPA (1,4,7,10,13-pentaazacyclopentadecane-N,N′,N″,N″′,N″″-pentaacetic acid); SarAr (1-N-(4-Aminobenzyl)-3,6,10,13,16,19-hexaazabicyclo[6.6.6]-eicosane-1,8-diamine) or a derivative thereof; NOTA (1,4,7-triazacyclononane-1,4,7-triacetic acid) or a derivative thereof; NETA (4-[2-(bis-carboxymethylamino)-ethyl]-7-carboxymethyl-[1,4,7]triazonan-1-yl) acetic acid or a derivative thereof; TRAP (1,4,7-triazacyclononane-1,4,7-tris[methyl(2-carboxyethyl)phosphinic acid) or a derivative thereof, HBED (N,N0-bis(2-hydroxybenzyl)-ethylenediamine-N,N0-diacetic acid) or a derivative thereof, 2,3-HOPO (3-hydroxypyridin-2-one) or a derivative thereof; PCTA (3,6,9,15-tetraazabicyclo[9.3.1]-pentadeca-1(15),11,13-triene-3,6,9,-triacetic acid) or a derivative thereof; PDTA (1,3-propylenediaminetetraacetate) or a derivative thereof;
NTA (nitrilotriacetate) or a derivative thereof, EDDS (ethylenediaminedisuccinate) or a derivative thereof; EDTA (ethylene diamine tetraacetic acid) or a derivative thereof; MGDA (N-(1-carboxylatoethyl)iminodiacetate) or a derivative thereof; DFO (desferrioxamine) or a derivative thereof; DTPA (diethylenetriaminepentaacetic acid) or a derivative thereof, CDTA ((1,2-cyclohexylenedinitrilo)tetraacetic acid) or a derivative thereof; CPTA (1,4,8,11-tetraazacyclotetradecane derivative) or a derivative thereof; OCTAPA (N,N0-bis(6-carboxy-2-pyridylmethyl)-ethylenediamine-N,N0-diacetic acid) or a derivative thereof, H2-MACROPA (N,N′-bis[(6-carboxy-2-pyridipmethyl]-4,13-diaza-18-crown-6) or a derivative thereof, H2dedpa (1,2-[[carboxy)-pyridin-2-yl]-methylamino]ethane or a derivative thereof, HEDP (hydroxyethylidene diphosphonate or etideronate) or a derivative thereof, HEBD (N,N′-bis(2-hydroxybenzyl) ethylenediamine-N,N′-diacetic acid) or a derivative thereof, HYNIC (6-Hydrazinopridine-3-carboxylic acid) or a derivative thereof; DMSA (meso-2,3-dimercaptosuccinic acid) or a derivative thereof; or EC20-head comprising β-1-diaminopropionic acid, Asp, and Cys.

The ligand and the payload, such as an imaging agent or an acrolein scavenger, of the conjugates can be linked by any suitable linker. The linker can be hydrophilic. The linker can comprise a hydrophilic amino acid, a polyethylene glycol polymer, a peptidoglycan, a sugar, or a combination of two or more of the preceding. The linker can comprise a hydrophilic amino acid, a polyethylene glycol polymer, or a combination thereof. The linker can comprise other hydrophilic biomolecules, including natural and unnatural biomolecules.

For a linker that comprises one or more PEG units, all carbon and oxygen atoms of the PEG units are part of the backbone unless otherwise specified. A cleavable bond for a releasable linker is part of the backbone. The “backbone” of the linker is the shortest chain of contiguous atoms forming a covalently bonded connection between the ligand and the payload. In some embodiments, a polyvalent linker has a branched backbone, with each branch serving as a section of backbone linker until reaching a terminus.

The linker can have any suitable length and chemical composition. For example, the linker can have a chain length of at least about 7 atoms in length. In some embodiments, the linker is at least about 10 atoms in length. In some embodiments, the linker is at least about 14 atoms in length. In some embodiments, the linker is between about 7 and about 31 (such as, about 7 to 31, 7 to about 31 or 7 to 31) between about 7 and about 24 (such as, about 7 to 24, 7 to about 24 or 7 to 24), or between about 7 and about 20 (such as, about 7 to 20, 7 to about 20 or 7 to 20) atoms in length. In some embodiments, the linker is between about 14 and about 31 (such as, about 14 to 31, 14 to about 31 or 14 to 31), between about 14 and about 24 (such as, about 14 to 24, 14 to about 24 or 14 to 24), or between about 14 and about 20 (such as, about 14 to 20, 14 to about 20 or 14 to 20) atoms in length. In some embodiments, the linker has a chain length of at least 7 atoms, at least 14 atoms, at least 20 atoms, at least 25 atoms, at least 30 atoms, at least 40 atoms; or from 1 to 15 atoms, 1 to 5 atoms, 5 to 10 atoms, 5 to 20 atoms, 10 to 40 atoms or 25 to 100 atoms.

The atoms used in forming the linker can be combined in all chemically relevant ways, such as chains of carbon atoms forming alkylene groups, chains of carbon and oxygen atoms forming polyoxyalkylene groups, chains of carbon and nitrogen atoms forming polyamines, and others. In addition, it is to be understood that the bonds connecting atoms in the chain may be either saturated or unsaturated, such that for example, alkanes, alkenes, alkynes, cycloalkanes, arylenes, imides, and the like may be divalent radicals that are included in the linker. In addition, it is to be understood that the atoms forming the linker may also be cyclized upon each other to form saturated or unsaturated divalent cyclic radicals in the linker.

Alternatively, or in addition to chain length, in some embodiments the linker has suitable substituents that can affect the hydrophobicity or hydrophilicity of the linker. Thus, for example, the linker can have a hydrophobic side chain group, such as an alkyl, cycloalkyl, aryl, arylalkyl, or like group, each of which is optionally substituted. If the linker were to include one or more amino acids, the linker can contain hydrophobic amino acid side chains, such as one or more amino acid side chains from phenylalanine (Phe) and tyrosine (Tyr), including substituted variants thereof, and analogs and derivatives of such side chains.

In some embodiments, the linker can comprise portions that are neutral under physiological conditions. In some embodiments, the linker can comprise portions that can be protonated or deprotonated to carry one or more positive or one or more negative charges, respectively. In some embodiments, the linker can comprise neutral portions and portions that may be protonated to carry one or more positive charges. Examples of neutral portions include poly hydroxyl groups, such as sugars, carbohydrates, saccharides, inositols, and the like, and/or polyether groups, such as polyoxyalkylene groups including polyoxyethylene, polyoxypropylene, and the like. Examples of portions that can be protonated to carry one or more positive charges include amino groups, such as polyaminoalkylenes including ethylene diamines, propylene diamines, butylene diamines and the like, and/or heterocycles including pyrrolidines, piperidines, piperazines, and other amino groups, each of which can be optionally substituted. Examples of portions that can be deprotonated to carry one or more negative charges include carboxylic acids, such as aspartic acid, glutamic acid, and longer chain carboxylic acid groups, and sulfuric acid esters, such as alkyl esters of sulfuric acid.

In some embodiments the linker can include at least one releasable portion. In some embodiments, the linker can include at least two releasable linkers (e.g., cleavable linkers). The choice of a releasable linker or a non-releasable linker can be made independently for each application or configuration of the compounds described herein. The releasable linkers described herein comprise various atoms, chains of atoms, functional groups, and combinations of functional groups. For example, in some embodiments the releasable linker comprises about 1 to about 30 atoms (e.g., about 1 to 30, 1 to about 30, and 1 to 30 atoms), or about 2 to about 20 atoms (e.g., about 2 to 20, 2 to about 20, and 2 to 20 atoms). Lower molecular weight linkers (e.g., those having an approximate molecular weight of about 30 g/mol to about 1,000 g/mol, such as from about 30 g/mol to about 300 g/mol, about 100 g/mol to about 500 g/mol or about 150 g/mol to about 600 g/mol) are also described. Precursors to such linkers may be selected to have either nucleophilic or electrophilic functional groups, or both, optionally in a protected form with a readily cleavable protecting group to facilitate their use in synthesis of the intermediate species.

The linker can be self-immolative. A self-immolative linker can release a payload in response to a stimulus as may, and can, be present in the CSF or CNS, such as at a site of neurotrauma (e.g., a spinal trauma (e.g., a spinal injury) or a brain trauma (e.g., a brain injury)), where there is inflammation, in a patient. Examples of such stimuli include, but are not limited to, pH, a redox system, a chemical trigger, an enzyme trigger, a 1,4-, 1,6-, or 1,8-elimination, a photodegradable trigger, or a combination of triggers, among others. A non-releasable linker can be used in place of a self-immolative linker, such as when a payload is an imaging agent.

In some embodiments, the conjugate includes a linker that releases the payload by a release mechanism involving reduction, oxidation, or hydrolysis. An example of a reduction mechanism includes reduction of a disulfide group into two separate sulfyhydryl groups.

An example of a self-immolative disulfide includes a sterically protected disulfide bond. The payload can be attached to the linker via any other suitable self-immolative bond, including via a self-immolative cathepsin cleavable amino acid sequence; via a self-immolative furin cleavable amino acid sequence; via a self-immolative β-glucuronidase cleavable moiety; via a self-immolative phosphatase cleavable moiety; or via a self-immolative sulfatase cleavable moiety.

In some embodiments, the linker comprises a phosphate or pyrophosphate group. In some embodiments, the linker comprises a cathepsin B cleavable group. In some embodiments, the cathepsin B cleavable group is Valine-Citrulline. In some embodiments, the linker comprises a carbamate moiety. In some embodiments, the linker comprises a β-glucuronide.

In some embodiments, the linker comprises an ester, phosphate, oxime, acetal, pyrophosphate, polyphosphate, disulfide, sulfate, hydrazide, imine, carbonate, carbamate or enzyme-cleavable amino acid sequence.

A releasable linker can comprise a disulfide group. Examples include divalent radicals comprising alkyleneaziridin-1-yl, alkylenecarbonylaziridin-1-yl, carbonylalkylaziridin-1-yl, alkylenesulfoxylaziridin-1-yl, sulfoxylalkylaziridin-1-yl, sulfonylalkylaziridin-1-yl, or alkylenesulfonylaziridin-1-yl groups, wherein each of the releasable linkers is optionally substituted. Additional examples include divalent radicals comprising methylene, 1-alkoxyalkylene, 1-alkoxycycloalkylene, 1-alkoxyalkylenecarbonyl, 1-alkoxycycloalkylenecarbonyl, carbonylarylcarbonyl,carbonyl(carboxyaryl) carbonyl, carbonyl(biscarboxyaryl)carbonyl, haloalkylenecarbonyl, alkylene(dialkylsilyl), alkylene(alkylarylsilyl), alkylene(diarylsilyl), (dialkylsilyl)aryl, (alkylarylsilyl)aryl, (diarylsilyl)aryl, oxycarbonyloxy, oxycarbonyloxyalkyl, sulfonyloxy, oxysulfonylalkyl, iminoalkylidenyl, carbonylalkylideniminyl, iminocycloalkylidenyl, carbonylcycloalkylideniminyl, alkylenethio, alkylenearylthio and carbonylalkylthio groups, wherein each of the releasable linkers is optionally substituted.

A releasable linker can comprise an oxygen atom and methylene, 1-alkoxyalkylene, 1-alkoxycycloalkylene, 1-alkoxyalkylenecarbonyl or 1-alkoxycycloalkylenecarbonyl groups, wherein each of the releasable linkers is optionally substituted. Alternatively, in some embodiments the releasable linker can include an oxygen atom and a methylene group, wherein the methylene group is substituted with an optionally substituted aryl, and the releasable linker is bonded to the oxygen to form an acetal or ketal. Further, in some embodiments the releasable linker can includes an oxygen atom and a sulfonylalkyl group, and the releasable linker is bonded to the oxygen to form an alkylsulfonate.

A releasable linker can include a nitrogen and iminoalkylidenyl, carbonylalkylideniminyl, iminocycloalkylidenyl, and carbonylcycloalkylideniminyl groups, wherein each of the releasable linkers is optionally substituted and the releasable linker is bonded to the nitrogen to form a hydrazone. In some embodiments, the hydrazone is acylated with a carboxylic acid derivative, an orthoformate derivative, or a carbamoyl derivative to form various acylhydrazone releasable linkers.

A releasable linker can include an oxygen atom and alkylene(dialkylsilyl), alkylene(alkylarylsilyl), alkylene(diarylsilyl), (dialkylsilyl)aryl, (alkylarylsilyl)aryl or (diarylsilyl)aryl groups, wherein each of the releasable linkers is optionally substituted and the releasable linker is bonded to the oxygen to form a silanol.

A releasable linker can include two independent nitrogens and carbonylarylcarbonyl, carbonyl(carboxyaryl)carbonyl, or carbonyl(biscarboxyaryl)carbonyl. In some embodiments the releasable linker can be bonded to the heteroatom nitrogen to form an amide, and also bonded to X^{a} or R^{a} via an amide bond.

A releasable linker can include an oxygen atom, a nitrogen, and a carbonylarylcarbonyl, carbonyl(carboxyaryl)carbonyl, or carbonyl(biscarboxyaryl)carbonyl. In some embodiments, the releasable linker forms an amide, and in some embodiments is bonded to X^{a} or R^{a} via an amide bond.

In some embodiments, the linker can comprise an optionally substituted 1-alkylenesuccinimid-3-yl group and a releasable portion comprising methylene, 1-alkoxyalkylene, 1-alkoxycycloalkylene, 1-alkoxyalkylenecarbonyl or 1-alkoxycycloalkylenecarbonyl groups, each of which can be optionally substituted, to form a succinimid-1-ylalkyl acetal or ketal.

In some embodiments, the linker can comprise carbonyl, thionocarbonyl, alkylene, cycloalkylene, alkylenecycloalkyl, alkylenecarbonyl, cycloalkylenecarbonyl, carbonylalkylcarbonyl, 1-alkylenesuccinimid-3-yl, 1-(carbonylalkyl)succinimid-3-yl, alkylenesulfoxyl, sulfonylalkyl, alkylenesulfoxylalkyl, alkylenesulfonylalkyl, carbonyltetrahydro-2H-pyranyl, carbonyltetrahydrofuranyl, 1-(carbonyltetrahydro-2H-pyranyl)succinimid-3-yl or 1-(carbonyltetrahydrofuranyl)succinimid-3-yl, each of which is optionally substituted. In some embodiments, the linker further comprises an additional nitrogen such that the linker comprises alkylenecarbonyl, cycloalkylenecarbonyl, carbonylalkylcarbonyl or 1-(carbonylalkyl)succinimid-3-yl groups, each of which is optionally substituted, bonded to the nitrogen to form an amide. In some embodiments, the linker further comprises a sulfur atom and alkylene or cycloalkylene groups, each of which is optionally substituted with carboxy, and is bonded to the sulfur to form a thiol. In some embodiments, the linker comprises a sulfur atom and 1-alkylenesuccinimid-3-yl and 1-(carbonylalkyl)succinimid-3-yl groups bonded to the sulfur to form a succinimid-3-ylthiol.

In some embodiments the linker comprises a nitrogen and a releasable portion comprising alkyleneaziridin-1-yl, carbonylalkylaziridin-1-yl, sulfoxylalkylaziridin-1-yl, or sulfonylalkylaziridin-1-yl, each of which is optionally substituted. In some embodiments, the linker comprises carbonyl, thionocarbonyl, alkylenecarbonyl, cycloalkylenecarbonyl, carbonylalkylcarbonyl, or 1-(carbonylalkyl)succinimid-3-yl, each of which is optionally substituted, and bonded to the releasable portion to form an aziridine amide.

The linker can have any suitable assortment of atoms in the chain, including C (e.g., —CH₂—, C(O)), N (e.g., NH, NR^{b}, wherein R^{b} is, e.g., H, alkyl, alkylaryl, and the like), O (e.g., —O—), P (e.g., —O—P(O)(OH)O—), and S (e.g., —S—). For example, the atoms used in forming the linker can be combined in all chemically relevant ways, such as chains of carbon atoms forming alkyl groups, chains of carbon and oxygen atoms forming polyoxyalkyl groups, chains of carbon and nitrogen atoms forming polyamines, and others, including rings, such as those that form aryl and heterocyclyl groups (e.g., triazoles, oxazoles, and the like). In addition, the bonds connecting atoms in the chain in the linker may be either saturated or unsaturated, such that for example, alkanes, alkenes, alkynes, cycloalkanes, arylenes, imides, and the like may be divalent radicals that are included in the linker. Further, the chain-forming linker can be substituted or unsubstituted.

Additional examples of linker groups include the groups 1-alkylsuccinimid-3-yl, carbonyl, thionocarbonyl, alkyl, cycloalkyl, alkylcycloalkyl, alkylcarbonyl, cycloalkylcarbonyl, carbonylalkylcarbonyl, 1-alkylsuccinimid-3-yl, 1-(carbonylalkyl)succinimid-3-yl, alkylsulfoxyl, sulfonylalkyl, alkylsulfoxylalkyl, alkylsulfonylalkyl, carbonyltetrahydro-2H-pyranyl, carbonyltetrahydrofuranyl, 1-(carbonyltetrahydro-2H-pyranyl)succinimid-3-yl, and 1-(carbonyltetrahydrofuranyl)succinimid-3-yl, wherein each group can be substituted or unsubstituted. Any of the aforementioned groups can be the linker or can be included as a portion of the linker. In some instances, any of the aforementioned groups can be used in combination (or more than once) (e.g., -alkyl-C(O)-alkyl) and can further comprise an additional nitrogen (e.g., alkyl-C(O)—NH—, —NH-alkyl-C(O)— or —NH-alkyl-), oxygen (e.g., -alkyl-O-alkyl-) or sulfur (e.g., -alkyl-S-alkyl-). Examples of such linker groups are alkylcarbonyl, cycloalkylcarbonyl, carbonylalkylcarbonyl, 1-(carbonylalkyl)succinimid-3-yl, and succinimid-3-ylthiol, wherein each group can be substituted or unsubstituted.

The linker can comprise an amino acid. In some embodiments, the linker can comprise an amino acid selected from the group consisting of Lys, Asn, Thr, Ser, Ile, Met, Pro, His, Gln, Arg, Gly, Asp, Glu, Ala, Val, Phe, Leu, Tyr, Cys, and Trp. In some embodiments, the linker can comprise at least two amino acids independently selected from the group consisting of Glu and Cys. In some embodiments, the linker can comprise Glu-Glu, wherein the glutamic acids are covalently bonded to each other through the carboxylic acid side chains. In some embodiments, such as when the payload is dimercaprol, the linker can comprise an unnatural amino acid, such as penicillamine. Given that dimercaprol comprises two thiol groups, each thiol group can be linked to a linker, such as a linker comprising an unnatural amino acid, e.g., penicillamine, in which case each linker is linked to the ligand via the unnatural amino acid, e.g., penicillamine. In some embodiments, penicillamine is represented as “pen.”

In some embodiments, the linker can comprise one or more spacer linkers. Spacer linkers can be hydrophilic spacer linkers comprising a plurality of hydroxyl functional groups. A spacer can comprise any stable arrangement of atoms. A spacer can comprise one or more L′. Each L′ is independently selected from the group consisting of an amide, ester, urea, carbonate, carbamate, disulfide, amino acid, amine, ether, alkyl, alkene, alkyne, heteroalkyl (e.g., polyethylene glycol), cycloakyl, aryl, heterocycloalkyl, heteroaryl, carbohydrate, glycan, peptidoglycan, polypeptide, or any combination thereof. In some embodiments, a spacer comprises any one or more of the following units: an amide, ester, urea, carbonate, carbamate, disulfide, amino acid, amine, ether, alkyl, alkene, alkyne, heteroalkyl (e.g., PEG), cycloakyl, aryl, heterocycloalkyl, heteroaryl, carbohydrate, glycan, peptidoglycan, polypeptide, or any combination thereof. In some embodiments, a spacer or L′ can comprises a solubility enhancer or PK/PD modulator. In some embodiments, a spacer can comprise a glycosylated amino acid. In some embodiments, a spacer can comprise one or more monosaccharide, disaccharide, polysaccharide, glycan, or peptidoglycan. In some embodiments, a spacer can comprise a releasable moiety (e.g., a disulfide bond, an ester, or other moieties that can be cleaved in vivo). In some embodiments, a spacer can comprise one or more units such as ethylene (e.g., polyethylene), ethylene glycol (e.g., PEG), ethanolamine, ethylenediamine, and the like (e.g., propylene glycol, propanolamine, propylenediamine). In some embodiments, a spacer comprises an oligoethylene, PEG, alkyl chain, oligopeptide, polypeptide, rigid functionality, peptidoglycan, oligoproline, oligopiperidine, or any combination thereof. In some embodiments, a spacer comprises an oligoethylene glycol or a PEG. A spacer can comprise an oligoethylene glycol. In some embodiments, a spacer comprises a PEG. In some embodiments, a spacer comprises an oligopeptide or polypeptide. In some embodiments, a spacer comprises an oligopeptide. In some embodiments, a spacer comprises a polypeptide. In some embodiments, a spacer comprises a peptidoglycan. In some embodiments, a spacer does not comprise a glycan. In some embodiments, a spacer does not comprise a sugar. In some embodiments, a rigid functionality is an oligoproline or oligopiperidine. In some embodiments, a rigid functionality is an oligoproline. In some embodiments, a rigid functionality is an oligopiperidine. In some embodiments, a rigid functionality is an oligophenyl. In some embodiments, a rigid functionality is an oligoalkyne. In some embodiments, an oligoproline or oligopiperidine has about two up to and including about fifty, about two to about forty, about two to about thirty, about two to about twenty, about two to about fifteen, about two to about ten, or about two to about six repeating units (e.g., prolines or piperidines).

The ligand can be any suitable ligand that can bind to folate receptor α (FRα) and/or FRβ, e.g., a ligand that can bind to FRα, FRβ, or both FRα and FRβ. The ligand can be a radical of a folate, a radical of an antifolate, or a radical of a folate analog. Examples of ligands include, but are not limited to, folate, folic acid, pteroic acid, or a derivative or analog of any of the foregoing. Examples of a reduced folate include, but are not limited to, 5-methyltetrahydrofolate (5-MTHF), 5-formyltetrahydrofolate (5-formyl-THF), 10-formyltetrahydrofolate (10-formyl-THF), a 5,10-methylenetetrahydrofolate (5,10-methylene-THF), a 5,10-methenyltetrahydrofolate (5,10-methenyl-THF), a 5,10-formiminotetrahydrofolate (5,10-formimino-THF), a 5,6,7,8-tetrahydrofolate (THF), and a dihydrofolic acid (DHF).

An antifolate binds to a folate receptor and antagonizes the biological action of folic acid or one of its naturally occurring forms. Examples of antifolates include, but are not limited to, methotrexate, pemetrexed, proguanil, pyrimethamine, raltitrexed, pralatrexate, and trimethoprim.

A folate analog can a pteroyl moiety or a pteroyl-amino acid moiety or a folate analog. In some embodiments, the ligand is a pteroyl-amino acid radical. In some embodiments, the ligand is a pteroyl-amino acid radical in which the amino acid is selected from the group consisting of aspartic acid, lysine, tyrosine, cysteine, threonine, serine, histidine, arginine, and an unnatural amino acid with a derivatizable moiety in the side chain.

A folate analog can be a pyrido[2,3-d]pyrimidine analog ligand, a functional fragment or analog thereof with an affinity (for example, and without limitation, a high specificity) for the folate receptor. For example, such folate analogs may have a relative affinity for binding folate receptor, such as folate receptor beta (FRβ) of about 0.01 or greater as compared to folic acid at a temperature about 20° C./25° C./30° C./physiological temperature.

While structures shown above may be represented as flat, one of ordinary skill in the art will appreciate that the ligands and conjugates represented above include stereoisomers, i.e., ligands and conjugates with identical structures but different configurations or spatial arrangements. Stereoisomerism is often due to chirality or “handedness,” i.e., the presence of right-handed (R) and left-handed (L) forms of drugs which are not superimposable mirror images (i.e., “enantiomers”). Chiral conjugates (or conjugates comprising chiral ligands, for example) can be administered as mixtures or single enantiomers, particularly if there are important differences in their activity and pharmacokinetics to be taken into account. It is intended that the above structural representations encompass single enantiomers and mixtures thereof.

The conjugates may contain one or more chiral centers or may otherwise exist as multiple stereoisomers, such as enantiomers, diastereomers, and enantiomerically or diastereomerically enriched mixtures. The conjugates may exist as geometric isomers. The conjugates may exist in un-solvated forms as well as solvated forms, including hydrated forms. In general, the solvated forms are equivalent to un-solvated forms. The conjugates may exist in multiple crystalline or amorphous forms. In general, all physical forms are equivalent for the uses contemplated. The formulae include pharmaceutically acceptable salts (e.g., acid addition and base salts), hydrates, and/or solvates.

One of ordinary skill in the art will further appreciate that the above ligands and conjugates can be “deuterated,” meaning one or more hydrogen atoms can be replaced with deuterium. As deuterium and hydrogen have nearly the same physical properties, deuterium substitution is the smallest structural change that can be made. Replacement of hydrogen with deuterium can increase stability in the presence of other drugs, thereby reducing unwanted drug-drug interactions, and can significantly lower the rate of metabolism (due to the kinetic isotope effect). By lowering the rate of metabolism, half-life can be increased, toxic metabolite formation can be reduced, and the dosage amount and/or frequency can be decreased.

Suitable acid addition salts are formed from acids which form non-toxic salts. Examples include, but are not limited to, acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-maysylate, nicotionate, nitrate, orotate, oxalate, palmitate, pamoate, phosphate/hydrogen phosphate/dihydrogen phosphate, saccharate, stearate, succinate, tartrate, tosylate, and trifluoroacetate salts.

Suitable base salts are formed from bases which form non-toxic salts. Illustrative examples include, but are not limited to, arginine, benzathine, calcium, choline, diethylamine, diolamine, glycine, lysine, magnesium, meglumine, olamine, potassium, sodium tromethamine, and zinc salts. Hemi-salts of acids and bases, such as hemisulphate and hemicalcium salts, also may be formed.

The conjugates can be synthesized in accordance with methods known in the art. Such methods are exemplified herein. In some embodiments, the linker can be formed via click chemistry/click chemistry-derived. Those of skill in the art understand that the terms “click chemistry” and “click chemistry-derived” generally refer to a class of small molecule reactions commonly used in conjugation, allowing the joining of substrates of choice with specific molecules. Click chemistry is not a single specific reaction but describes a way of generating products that follow examples in nature, which also generates substances by joining small modular units. In many applications, click reactions join a biomolecule and a reporter molecule. Click chemistry is not limited to biological conditions: the concept of a “click” reaction has been used in pharmacological and various biomimetic applications. However, they have been made notably useful in the detection, localization and qualification of biomolecules. Click reactions can occur in one pot, typically are not disturbed by water, can generate minimal byproducts, and are “spring-loaded” characterized by a high thermodynamic driving force that drives it quickly and irreversibly to high yield of a single reaction product, with high reaction specificity (in some cases, with both regio- and stereo-specificity). These qualities make click reactions suitable to the problem of isolating and targeting molecules in complex biological environments. In such environments, products accordingly need to be physiologically stable and any byproducts need to be non-toxic (for in vivo systems).

The conjugates can be administered in a pharmaceutical composition, such as a pharmaceutical composition comprising a pharmaceutically acceptable carrier. The carrier can be an excipient. The choice of carrier can depend on factors such as the particular mode of administration, the effect of the carrier on solubility and stability, and the nature of the dosage form. The pharmaceutical composition can be formulated using routine methods known in the art. See, e.g., Remington: The Science & Practice of Pharmacy, 21^{st} edition (Lippincott Williams & Wilkins (2005)).

In view of the above, also provided is a pharmaceutical composition comprising (i) a conjugate comprising (a) a ligand, which binds FRα and/or FRβ, (b) a linker, and (c) an acrolein scavenger and (ii) a pharmaceutically acceptable carrier. The linker links the ligand to the acrolein scavenger.

### EXAMPLES

The following examples serve to illustrate the present disclosure. The examples are not intended to limit the scope of the claimed invention in any way.

### Example 1: Synthesis

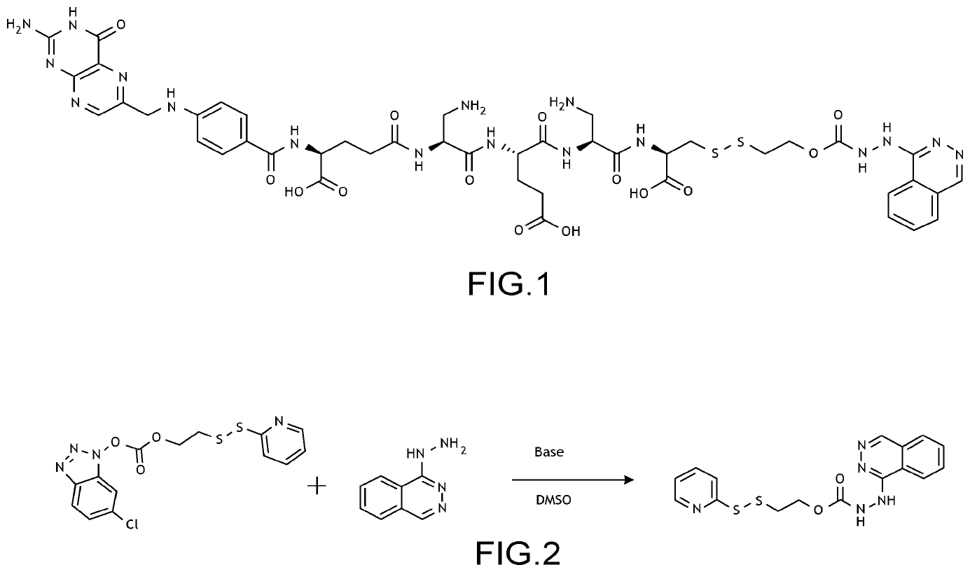

An example of a conjugate is shown in FIG. 1, which shows the structure of a conjugate comprising folate, a hydrophilic linker, and hydralazine. The folate is covalently linked to the hydrophilic linker, which comprises alternating unnatural amino acids D-glutamic acid and Dap and the natural amino acid cysteine. The cysteine is connected to hydralazine via a disulfide carbamate, self-immolative linker.

The reaction of the activation of hydralazine for linkage to folate is shown in FIG. 2. Briefly, 1 equivalent of 6-chloro-1H-benzo[d][1,2,3]triazol-1-yl (2-(pyridine-2-yldisulfaneyl)ethyl) carbonate was mixed with 1 equivalent of hydralazine in dimethylsulfoxide (DMSO) at room temperature. The product was purified by flash chromatography and verified by liquid chromatography-mass spectrometry (LC-MS).

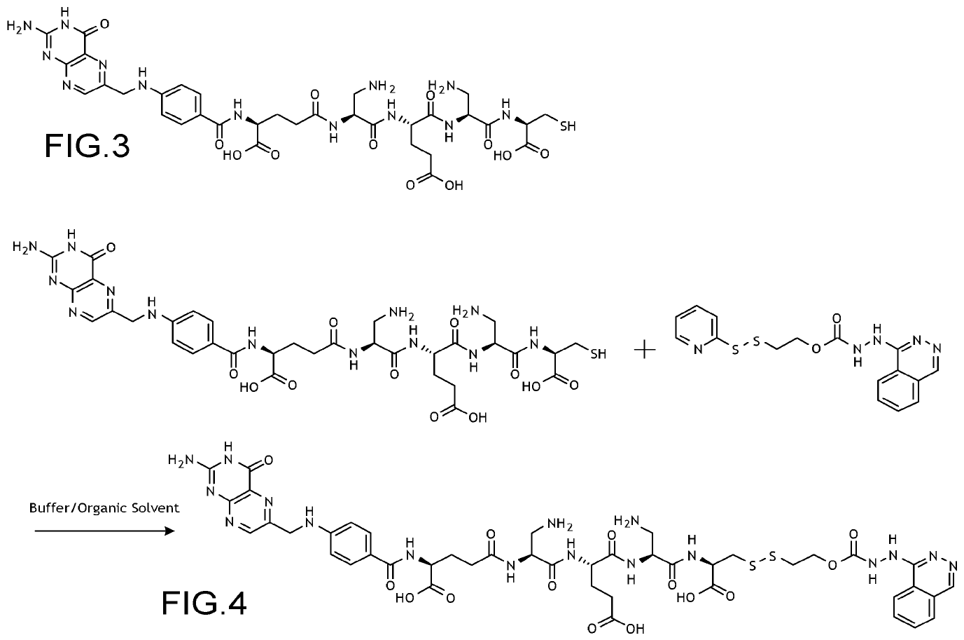

The structure of a folate-linker is shown in FIG. 3. The folate linker was synthesized via solid phase peptide synthesis (SPPS) using standard SPPS protocols, along with N,N,N′,N′-tetramethyl-O-(1H-benzotriazol-1-yl)uronium hexafluorophosphate (HBTU)/1-[Bis(dimethylamino)methylene]-1H-1,2,3-triazolo[4,5-b]pyridinium 3-oxid hexafluorophosphate (HATU) and the coupling reagent. Folate was coupled last via production of standard N,N′-dicyclohexylcarbodiimide (DCC) N-hydroxysuccinimide (NHS) by protocols known in the literature. The product was cleaved from the resin by TFA/TIPS/TCEP and purified by diethyl ether precipitation and verified by LC-MS.

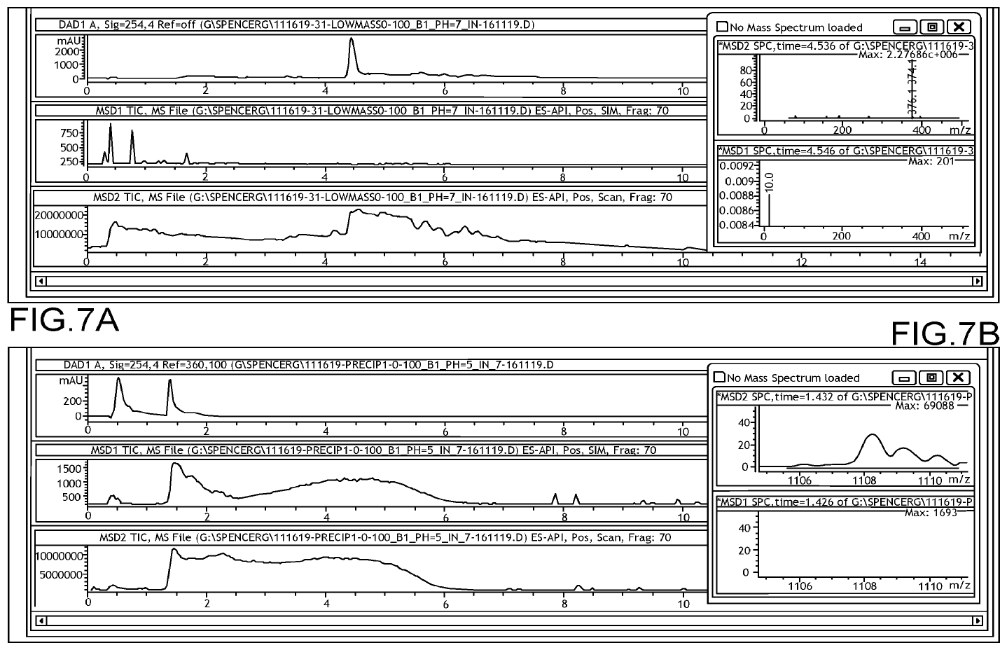

The reaction of a folate-linker with activated hydralazine is shown in FIG. 4. Briefly, 1 equivalent of folate-linker was dissolved in ammonium acetate buffer, pH 6.5. Then the activated hydralazine (1 eq) was dissolved in tetrahydrofuran (THF). The THF was added to the buffer containing the folate-linker and allowed to stir at room temperature overnight. The reaction was then purified by reverse-phase high-pressure liquid chromatography (RP-HPLC) and verified by LC-MS (see FIGS. 7A and 7B).

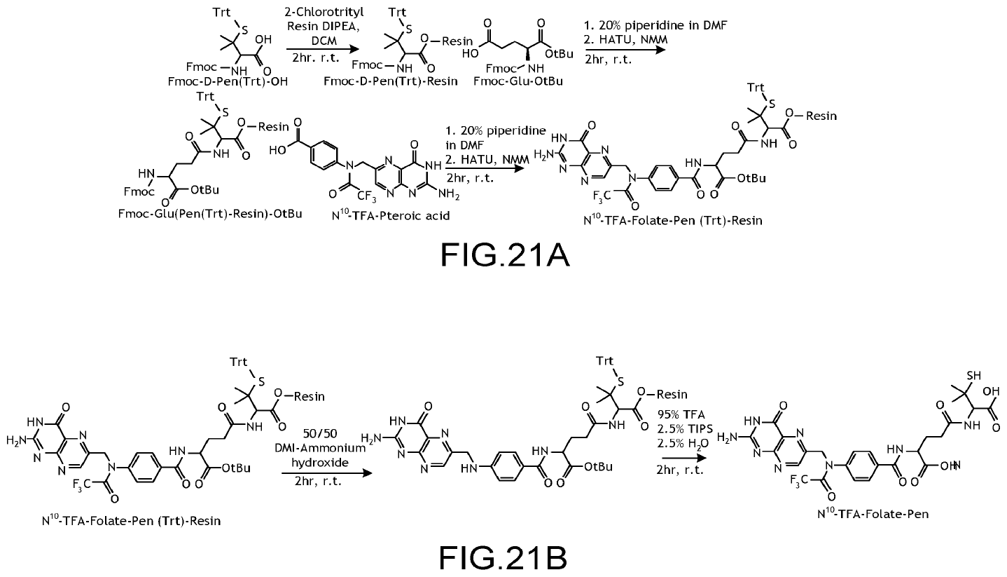

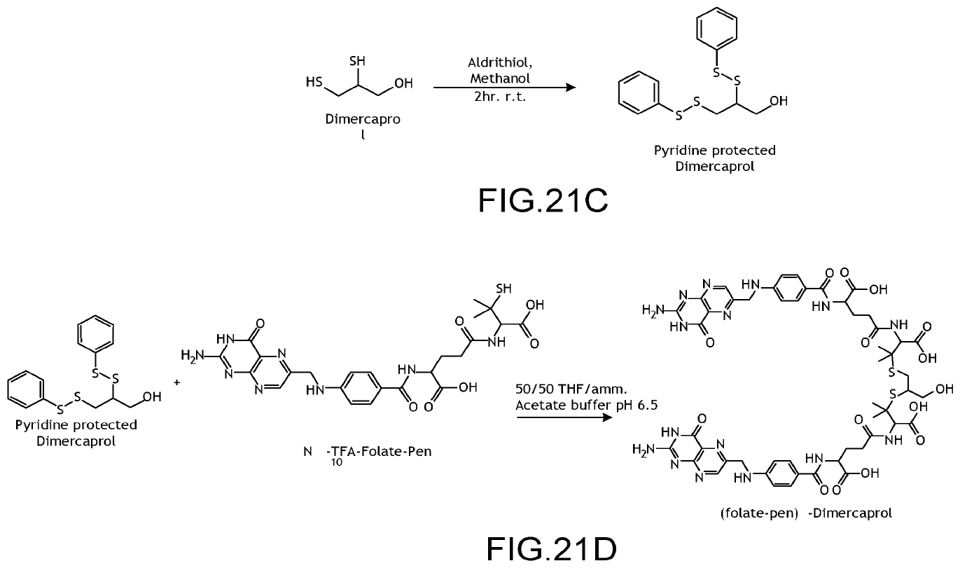

A reaction scheme for the synthesis of folate(2)-dimercaprol is shown in FIGS. 21A-21D.

### Example 2: Blood Pressure Data

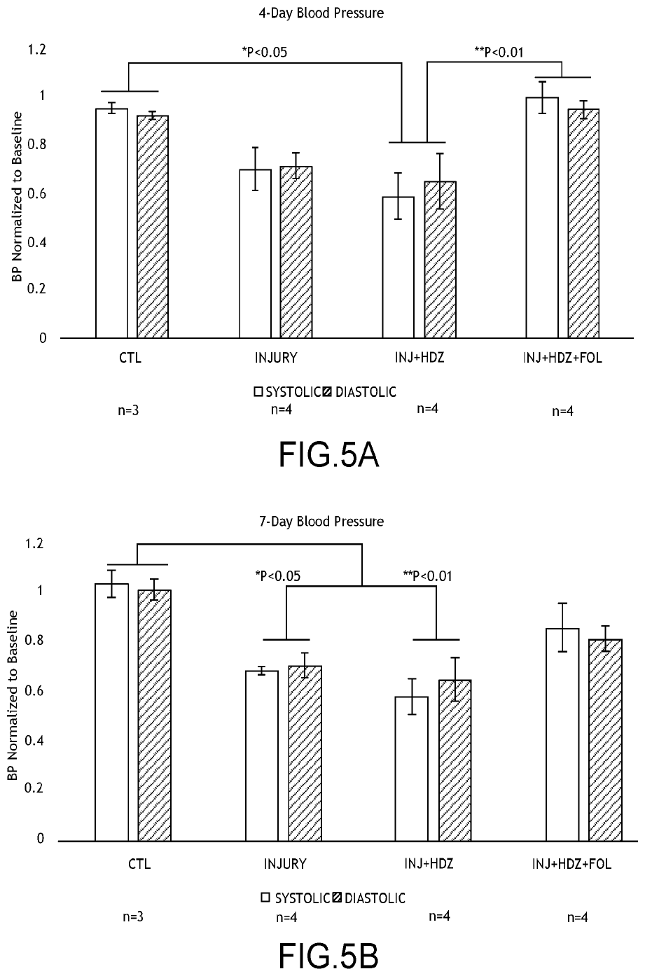

Baseline blood pressure in rats was recorded upon arrival in the laboratory. After acclimation (approximately two weeks) to the blood pressure set-up (CODA tail cuff system) and handling by lab personnel, the rats were given T10 spinal cord contusion injuries. After injury, the rats received daily tail intravenous injections of either 5 mg/kg hydralazine or a conjugate comprising folate-hydralazine. Blood pressure was recorded approximately 30 min post-injection on days 4 and 7 post-injury. Recordings were normalized to baseline values. ANOVA was used for statistics. The results are shown in FIGS. 5A-FIG. 5B, which are graphs of control (CTL), spinal injury (INJURY), spinal injury treated with hydralazine (INJ+HDZ), and spinal injury treated with a conjugate comprising folate linked to hydralazine (INJ+HDZ-FOL) vs. blood pressure (BP) normalized to baseline for systolic and diastolic blood pressure measured on day 4 (FIG. 5A) and day 7 (FIG. 5B).

### Example 3: Acrolein-Scavenging Data

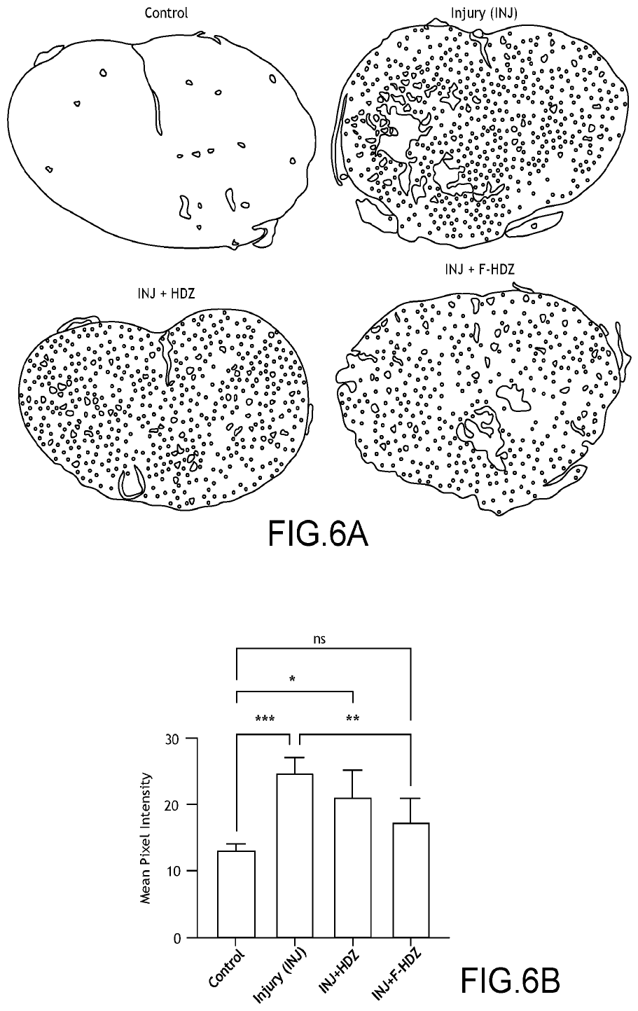

On day 8, after a final injection of drug, the rats from Example 2 were perfused with chilled phosphate-buffered saline (PBS). Spinal cords were dissected and stored in 4% paraformaldehyde (PFA) followed by cryoprotection in 30% sucrose and freezing in optimal temperature compound (OTC). OTC tissue cubes were sliced using a cryostat at 25 m thickness. Injury epicenter tissue was stained with an antibody against FDP-lysine (acrolein-bound lysine) and developed using standard diaminobenzidine-biotin (DAB-biotin) immunohistochemistry. Snapshots of relevant injury areas were taken and mean pixel intensity of tissue staining quantified using imageJ. ANOVA was used for statistics. Results are shown in FIGS. 6A-6B. FIG. 6B is a graph of control, injury (INJ), spinal injury treated with hydralazine (INJ+HDZ), and spinal injury treated with a conjugate comprising folate linked to hydralazine (INJ+HDZ-FOL) vs. acrolein levels detected by immunohistochemistry and expressed as mean pixel intensity. ns=not significant. *=P<0.10. **=P<0.01. ***=P<0.001. Images in FIG. 6A are from rat spinal cords on day 8 post spinal injury (HDZ=hydralazine. F-HDZ=folate-hydralazine).

### Example 4: Transport of Folate Conjugates Across Blood-Cerebrospinal Fluid Barrier (BCSFB)

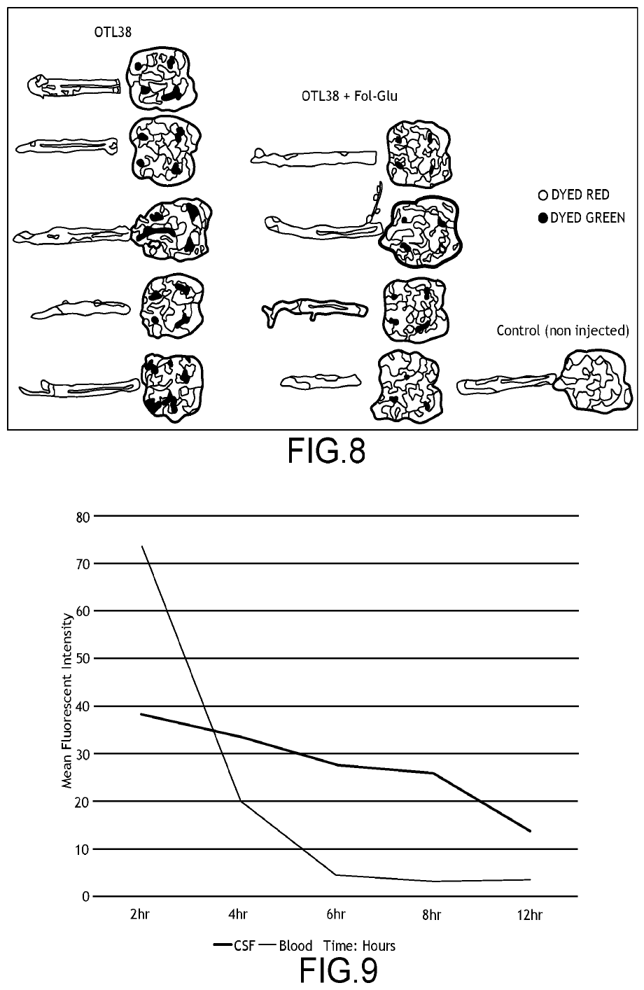

B6 male mice were given an intravenous tail injection of a folate-fluorescent near infrared (NIR) dye conjugate (i.e., OTL38) and imaged. The results are shown in FIG. 8, which shows images of brains divided in half with a single sagittal cut through the corpus collosum, revealing the choroid plexus in the left and right hemispheres. Corresponding ventral side of the spinal cords are shown to the left of each brain. Red autofluorescence reveals the tissue structure. Green fluorescence indicates OTL38 signal. The left column (OTL38, n=5) reveals strong signal in the choroid plexus. In the middle column (OTL38+Fol-Glu, n=4) minimal uptake is observed. In the far right corner, Control (non-injected, n=1), no signal is observed. Strong signal in the choroid plexus was observed 24 hours after injection. When mice were co-injected with OTL38 and a folate-glucosamine conjugate (Fol-Glu; a competitive agonist for folate receptors, such as FRα), a noticeable reduction in signal was observed. This indicates that the uptake of OTL38 is mediated by folate receptors on cells of the choroid plexus. No signal was observed in a control mouse, indicating that the choroid plexus does not auto-fluoresce in the NIR spectrum and the signals observed for mice injected with OTL38 is a result of OTL38 uptake.

### Example 5: Persistence of Folate Conjugates Transcytosed Across BCSFB

Adult male Sprague Dawley rats were injected with OTL38. Blood and cerebrospinal fluid (CSF) were then extracted at the same time at various timepoints and imaged. At two hours post-injection, the NIR signal in the blood exceeded that of the CSF by nearly two-fold. By four hours post-injection, the NIR signal in the blood dropped and remained lower than that of the CSF for all subsequent timepoints. These data indicate that the folate receptor exosome system (FRES) co-transports a payload delivered by conjugation to a folate ligand for release into the CSF. The data also indicate that the exposure of the CNS to the payload is extended over time.

The results are shown in FIG. 9. FIG. 9 is a graph of mean fluorescent intensity vs. time for CSF and blood. n=1-4 rats/timepoint.

### Example 6: FRES Uptake of Folate Conjugates in Spinal Cord Contusion Injury (SCI)

Neurotrauma is hallmarked by high levels of inflammation, including IBA1+ macrophages/microglial cells. These cells also express folate receptors, such as FRα and FRβ.

Adult male Sprague Dawley rats were given a moderate grade spinal cord contusion injury (SCI). The rats were injected with OTL38 or simultaneously with OTL38 and Fol-Glu on day 7 after injury. On day 8, the spinal cords were removed and imaged for NIR signal. Strong signal in the spinal cords of rats treated with OTL38 indicates targeting is due to FRES uptake by abundant folate receptor-positive cells, in contrast to the lack of signal in the spinal cords of rats treated with OTL38+Fol-Glu.

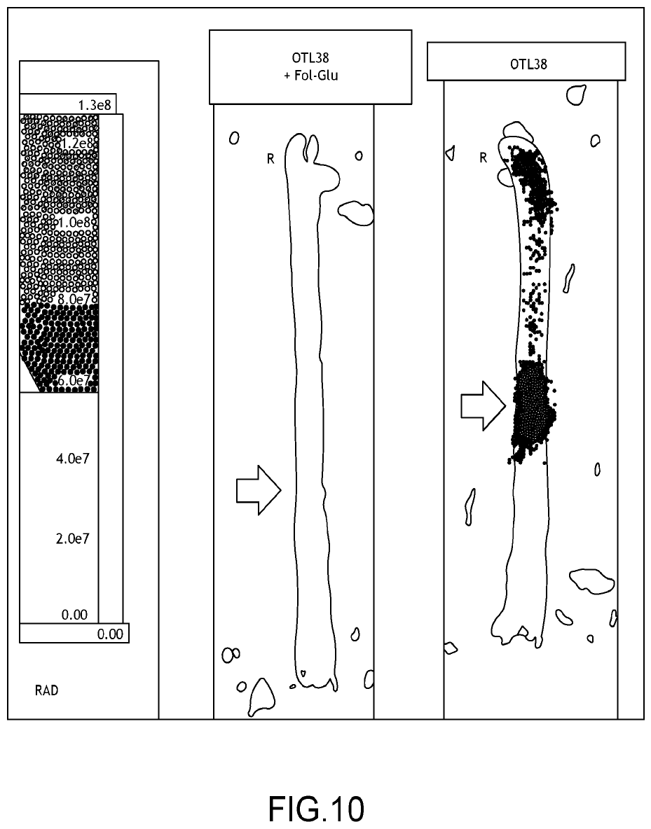

The results are shown in FIG. 10. FIG. 10 shows ex vivo imaging of the spinal cords of the rats. NR signal is represented by green and red colors, with red showing areas of high intensity. Blue arrows point to the epicenter of the SCI. “R” indicates the rostral portion of the spinal cord.

### Example 7: FRES Uptake of Folate Conjugates in SCI

Adult male Sprague Dawley rats were given a moderate grade SCI. The rats were injected with OTL38 or unconjugated NIR dye on day 7 after injury. On day 8, the spinal cords were removed and imaged for NIR signal. Signal was observed in the spinal cords of rats treated with OTL38, indicating targeting is due to FRES uptake by abundant folate receptor-positive cells, in contrast to the lack of signal in the spinal cords of rats treated with unconjugated NIR dye.

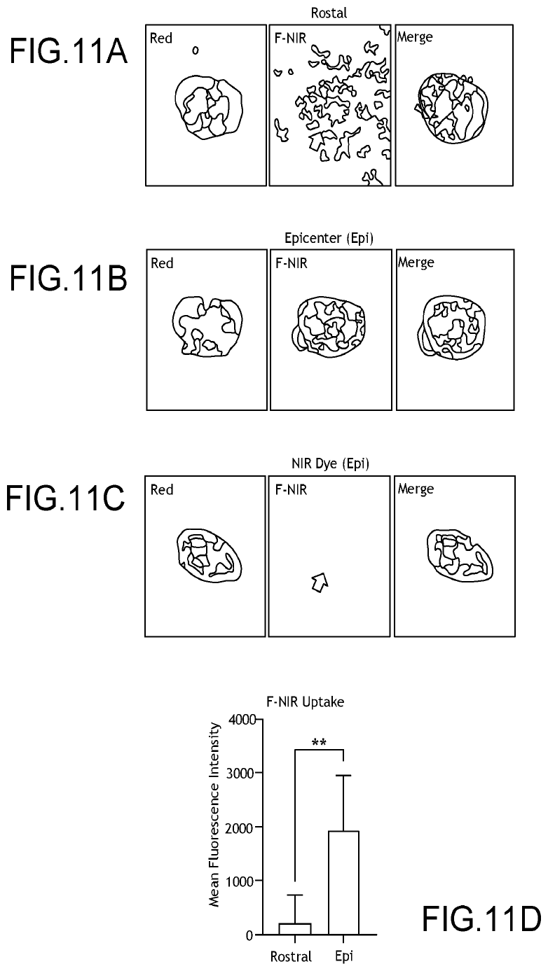

The results are shown in FIGS. 11A-11D. FIGS. 11A-11B show transverse spinal cord sections of the rostral portions (FIG. 11A) and epicenters (FIG. 11B) of a rat with SCI and injected seven days after injury with OTL38. FIG. 11C shows transverse spinal cord sections of the epicenters of a rat with SCI and injected seven days after injury with S0456 the unconjugated near infrared dye (NIR) dye S0456. The regions in FIGS. 11A-11B were quantified, revealing a significant increase in signal in the epicenter as compared to the rostral region. The results are shown in FIG. 11D, which is a bar graph of mean fluorescence intensity vs. rostral portion and epicenter showing OTL38 uptake (students t-test, **p<0.01). Red autofluorescence reveals tissue structure.

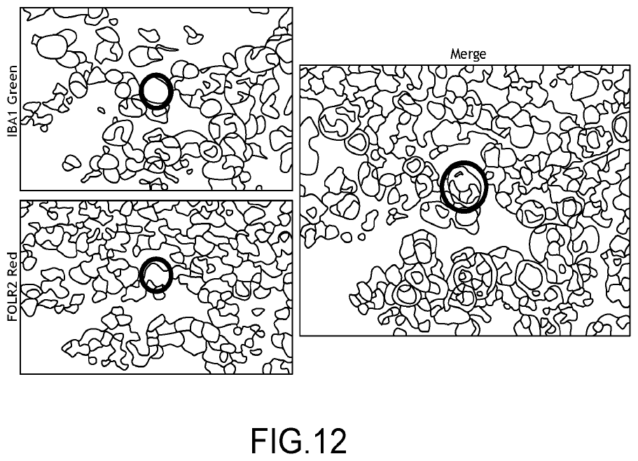

Transverse sections of the epicenters were stained using standard immunofluorescence techniques for IBA1-positive macrophage/microglia (green) and folate receptor β (FRβ; red). Images were taken using 10× fluorescent microscopy. Strain staining for IBA1 and FRβ was observed in the epicenters, as well as multiple instances of co-localization. The results are shown in FIG. 12, which shows transverse sections of SCI rat epicenter regions stained and imaged using 10× immunofluorescent microscopy with anti-IBA1 staining shown in green and anti-FRβ staining shown in red. The merge shows instances of co-localization, with one instance encircled in yellow. The data indicate multiple folate receptor types, including FRβ, and immune cells such as macrophage/microglia play a role in FRES targeting to CNS disease.

### Example 8: FRES Uptake of Folate Conjugates in Cortex Laceration

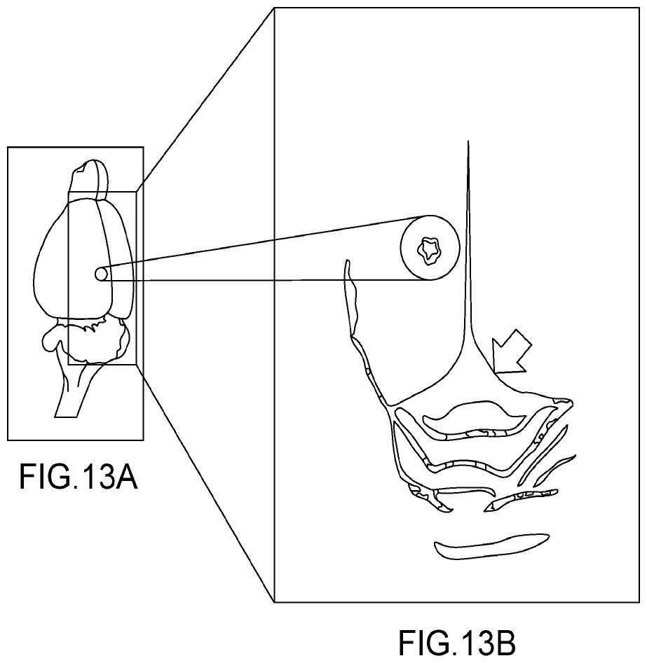

Adult male Sprague Dawley rats were given brain injuries by cortex laceration of the left hemisphere using a small ophthalmic knife. Seven days after injury, the rats were injected with OTL38. The brains were removed the next day, and the cortexes were imaged. The results are shown in FIGS. 13A-13B.

FIGS. 13A-13B show a precise surface laceration injury in the cortex of the left hemisphere of the brain. FIG. 13A is a cartoon indicating the area of injury (blue circle) and the area imaged (blue rectangle). FIG. 13B is an image showing enhanced signal around the injury area (blue circle). Increased signal around the choroid plexus is indicated with the blue arrow. The right hemisphere cortex served as an internal control (uninjured, no signal).

Choroid plexus cells in the ventricles produce CSF and are the site of FRES release into the subarachnoid space, where the brain and spinal cord continue to be perfused. The 4^{th} ventricle and lateral recess contain choroid plexus cells and are the site of outflow for newly produced CSF. The intense signal below the cerebellum (blue arrow) is a result of concentrated choroid plexus cells and FRES/new CSF production and entry for CNS perfusion. Successful targeting of the cortex injury is demonstrated by a precise, well-defined green spot (blue circle). The opposite hemisphere represents an internal control with no areas of enhanced signal.

### Example 9: FRES Uptake of Folate Conjugates in Cortex Impaction

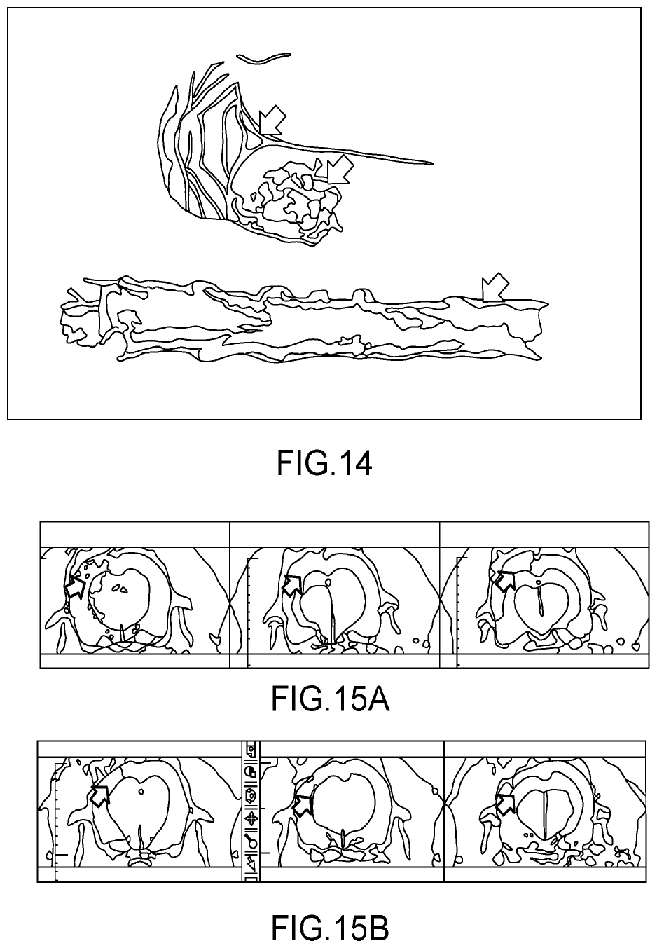

Adult male Sprague Dawley rats were given moderate grade controlled cortical impact (CCI) injuries. This injury represents concussion of the brain cortex, which is a more common injury in humans than laceration of the brain cortex. The impactor was circular and 6 mm in diameter. The rats were intravenously injected with OTL38 ten days after injury. The next day the rats were sacrificed, and the surfaces of the cortexes were imaged. FIG. 14 shows images of the brain and spinal cord of a rat with a CCI injury and injected with OTL38. Increased signal around the choroid plexus and FRES entry to the CNS/CSF is indicated with the blue arrow. The red arrow shows positive targeting of the 6 mm circular injury. The yellow arrow points to signal on the dorsal surface of the spinal cord. Strong signal was not observed in the left hemisphere (internal control).

In a separate experiment, adult male Sprague Dawley rats with CCI injuries were intravenously injected with the magnetic resonance imaging (MRI) contrast agent folate-gadolinium (fol-Gad) 10 days after injury. The next day the rats were scanned by T2 MRI. The results are shown in FIGS. 15A-15B. FIG. 15A shows MRI at various sections of a rat with a CCI injury and injected to fol-Gad. Blue arrows point to brain injury with enhanced contrast, and corresponding transverse section bregmas are shown at the tops of the images in millimeters. FIG. 15B shows MRI at various sections of a rat with a CCI injury and not injected with fol-Gad. Red arrows point to brain injury with no obvious enhancement in signal, and corresponding transverse section bregmas are shown at the tops of the images in millimeters. The results indicate additional FRES payloads, such as fol-Gad, have utility for neuroimaging and CNS targeting.

### Example 10: Delivery of Folate-S0456 Conjugates to Eye

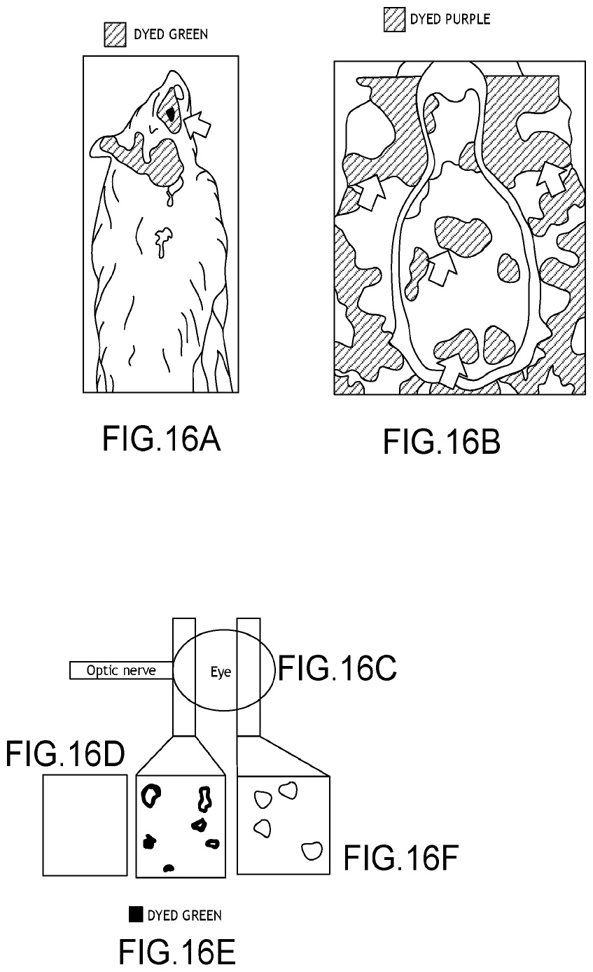

An adult male Sprague Dawley rat was intravenously injected with OTL38 and imaged 24 hours later. The results are shown in FIG. 16A. FIG. 16A shows the imaging of the live rat injected with OTL38 24 hours earlier. Red and green colors indicate signal from the dye, with red showing the most intense regions. The blue arrow indicates intense red signal at the eye.

An adult male Sprague Dawley rat was given a closed head injury and 48 hours later was intravenously injected with folate conjugated with the imaging agent technetium (Fol-Tc). The next day the rat was imaged with PET/CT. The results are shown in FIG. 16B. FIG. 16B shows the PET/CT scan of the rat with the closed head injury and injected with Fol-Tc. The purple color indicates signal from Fol-Tc uptake. The Blue arrows show intense signal around the retinal areas of the eye. The yellow arrows indicate the choroid plexus. Other signal is likely due to inflammation from the head injury. FIG. 16C is a diagram of where sections from the eye were sliced and analyzed for the images shown in FIG. 16D shows an image of a rate injected with unconjugated NIR dye. FIGS. 16E-16F show further images evidencing strong OTL38 uptake in the area of the retina. FRα is abundantly expressed in the ganglion cell layer, retinal pigment epithelium, inner nuclear cell layer, especially Müller cells, and inner segments of the photoreceptor cell layer. FIG. 16E shows an image of a slice taken from the retinal area nearest the optic nerve (corresponding to the red box in FIG. 16C) of a rat injected with OTL38. FIG. 16F shows an image of a slice taken from the area closer to the cornea and corresponding to the black box in FIG. 16C of the same rat injected with OTL38. Taken together, these data show evidence that folate conjugates can be delivered to the eye, either through the blood retina barrier and/or from contact with CSF and FRES at the optic nerve head/lamina cribosa layer. These data also indicate that additional payloads, such as Fol-TC, can be delivered to the CNS.

### Example 11: Retention of Folate Conjugates in Eye

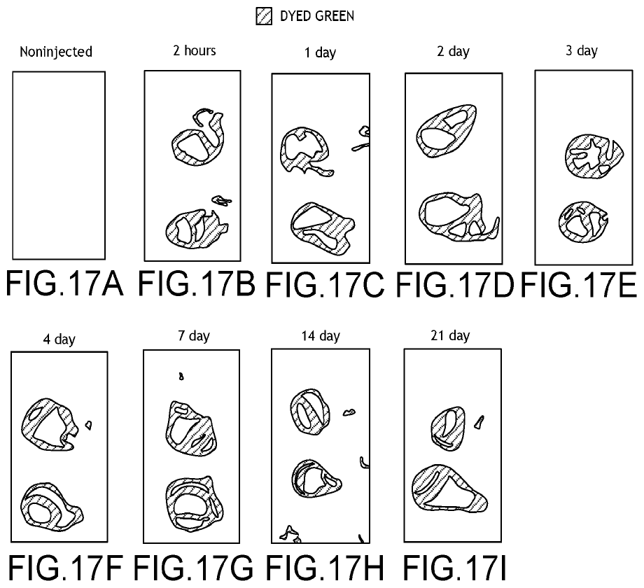

Live, healthy mice were injected intravenously with 20 nmols of folate-conjugated NIR dye (OTL38). Whole eyes were removed and scanned at various timepoints—2 hours or 1, 2, 3, 4, 7, 14 or 21 days post-injection. The results are shown in FIGS. 17A-I. FIGS. 17A-17I are images of scans of whole eyes removed from mice at various timepoints (2 hours or 1, 2, 3, 4, 7, 14 or 21 days) after intravenous injection with OTL38 as compared to control mice, which did not receive an injection of OTL38. Signal persisted up to 21 days.

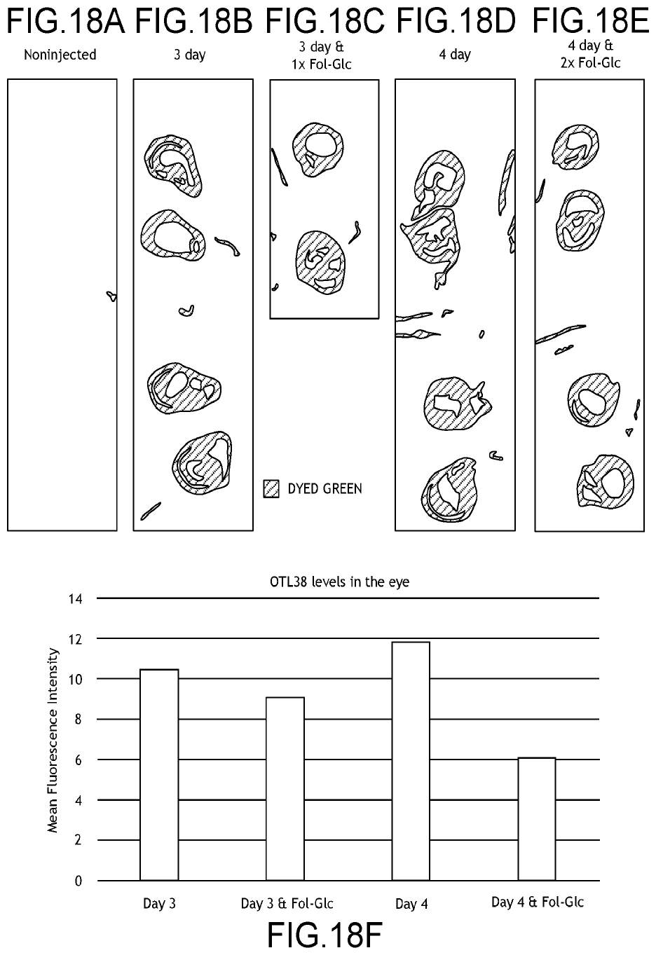

Given the extensive time that OTL38 remained in the eyes, signal in the presence of the folate competitor folate-glucosamine (Fol-Glc) was tested. Healthy, wild-type mice were injected with 10 nmols OTL38. Some mice were subsequently injected with Fol-Glc. All mice were subsequently sacrificed and examined in comparison to noninjected mice. The results are shown in FIGS. 18A-18E. Images of eyes of noninjected mice are shown in FIG. 18A. Images of eyes of mice injected with OTL38 and sacrificed three days later are shown in FIG. 18B, whereas images of eyes of mice injected with OTL38 on day 1, injected with 10 mmol Fol-Glc on day 2, and sacrificed on day 3 are shown in FIG. 18C, images of eyes of mice injected with OTL38 on day 1 and sacrificed on day 4 are shown in FIG. 18D, and images of eyes of mice injected with OTL38 on day 1, injected with 10 mmole Fol-Glc on days 2 and 3, and sacrificed on day 4 are shown in FIG. 18E. OTL38 signal was observed in all mice except the noninjected mice. The OTL38 signal, however, was reduced by administration of Fol-Glc as shown in FIG. 18F, which is a graph of treatment vs. mean fluorescence intensity.

### Example 12: Delivery of Folate-Cy5 Conjugate to Eye

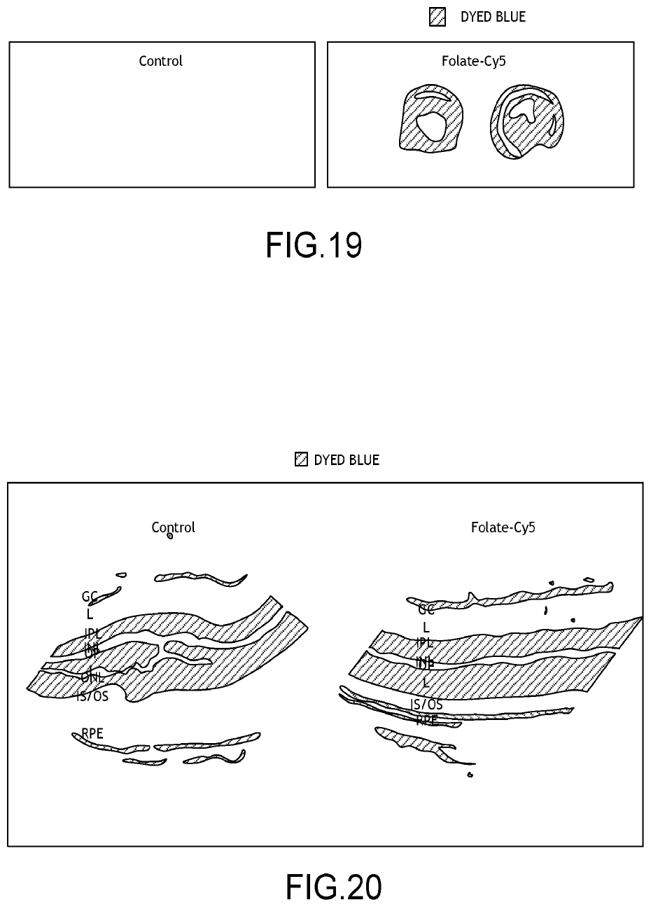

Mice were intravenously injected with 10 nmol of folate-Cy5. Whole eyes were extracted and imaged with a tissue imager 24 hours post-injection. While eyes extracted from treated mice showed a blue signal indicative of Cy5, eyes extracted from nontreated mice did not show any signal. FIG. 19 shows the images of eyes extracted from treated mice (Folate-Cy5) compared to images of eyes extracted from nontreated mice.

In a separate experiment, mice were intravenously injected three times with 10 nmol of folate-Cy5 over a 7-day period. Whole eyes were extracted, co-stained with nuclear stain DAPI (blue), cryo-sectioned, and imaged. While eyes extracted from treated mice showed a purple signal indicative of Cy5, eyes extracted from nontreated mice did not show any signal. FIG. 20 shows the images of eyes extracted from treated mice (Folate-Cy5) compared to images of eyes extracted from nontreated mice. Layers of the eye are labeled: GCL=ganglion cell layer; IPL=inner plexiform layer; INL=inner nuclear layer; OPL=outer plexiform layer; ONL=outer nuclear layer; IS/OS=inner and outer segments of the photoreceptor layer; RPE=retinal pigmented epithelium. The RPE shows strong signal for folate-Cy5, indicating targeting of these cell types by folate conjugates.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

The invention illustratively described herein may be suitably practiced in the absence of any element(s) or limitation(s), which is/are not specifically disclosed herein. Thus, for example, each instance herein of any of the terms “comprising,” “consisting essentially of,” and “consisting of” may be replaced with either of the other two terms. Likewise, the singular forms “a,” “an,” and “the” include plural references unless the context clearly dictates otherwise. Thus, for example, references to “the method” includes one or more methods and/or steps of the type, which are described herein and/or which will become apparent to those ordinarily skilled in the art upon reading the disclosure.

The terms and expressions, which have been employed, are used as terms of description and not of limitation. Where certain terms are defined and are otherwise described or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings may be used in the “Detailed Description,” such use is solely for ease of reference and is not intended to limit any disclosure made in one section to that section only; rather, any disclosure made under one subheading is intended to constitute a disclosure under each and every other subheading.

It is recognized that various modifications are possible within the scope of the claimed invention. Thus, although the present invention has been specifically disclosed in the context of preferred embodiments and optional features, those skilled in the art may resort to modifications and variations of the concepts disclosed herein. Such modifications and variations are considered to be within the scope of the invention as claimed herein.

1. A method of delivering a payload to a patient with inflammation in the central nervous system (CNS), which method comprises administering to the patient with inflammation in the CNS an effective amount of a conjugate comprising:
(a) a ligand, which binds folate receptor α (FRα) and/or folate receptor β (FRβ),
(b) a payload selected from an acrolein scavenger and an imaging agent, and
(c) a linker, which links the ligand and the payload.

2. The method of claim 1, wherein the ligand is a folate or a derivative or analog thereof.

3. The method of claim 1, wherein the ligand is 5-methyltetrahydrofolate (5-MTHF).

4. The method of claim 1, wherein the linker is hydrophilic or self-immolative.

5. (canceled)

6. The method of claim 1, wherein the linker comprises a hydrophilic amino acid, a polyethylene glycol polymer, a peptidoglycan, a sugar, or a combination of two or more of the preceding.

7. (canceled)

8. The method of claim 1, wherein the imaging agent is a near infrared (NIR) dye.

9. The method of claim 8, wherein the NIR dye is S0456 or Cy5.

10. The method of claim 1, wherein the conjugate is OTL38.

11. The method of claim 1, wherein the imaging agent is a magnetic resonance imaging (MRI) contrast agent or a positron emission tomography/computed tomography (PET/CT) contrast agent.

12. The method of claim 11, wherein the MRI contrast agent is gadolinium or the PET/CT contrast agent is technetium.

13. The method of claim 1, which further comprises imaging an area of the CNS patient affected by the inflammation.

14-15. (canceled)

16. The method of claim 1, wherein the payload is transported to an eye.

17. The method of claim 1, wherein the acrolein scavenger is hydralazine or dimercaprol, wherein, when the acrolein scavenger is dimercaprol, each thiol group in dimercaprol is linked to a linker, which is linked to a ligand.

18. The method of claim 17, wherein each thiol group in dimercaprol is linked to a linker comprising a penicillamine and the linker is attached to the ligand by the penicillamine.

19-22. (canceled)

23. The method of claim 1, wherein the patient has neurotrauma, stroke, epilepsy, multiple sclerosis, motor neuron disease, a movement disorder, Alzheimer's disease, amyotrophic lateral sclerosis, Creutzfeldt-Jakob disease, depression, anxiety, schizophrenia, Guillain-Barré syndrome, an infection or inflammatory disease of the central nervous system, optic atrophy, retinal degeneration, ocular proptosis, ocular trauma, an ocular inflammatory disorder, glaucoma, or macular degeneration.

24. The method of claim 23, wherein the movement disorder is Parkinson's disease.

25. A conjugate comprising:
(a) a ligand, which binds folate receptor α (FRα) and/or folate receptor β (FRβ),
(b) a linker, and
(c) an acrolein scavenger,
wherein the linker links the ligand and the acrolein scavenger.

26. The conjugate of claim 25, wherein the ligand is a folate or a derivative or analog thereof.

27. The conjugate of claim 25, wherein the ligand is 5-methyltetrahydrofolate (5-MTHF).

28. The conjugate of claim 25, wherein the acrolein scavenger is hydralazine or dimercaprol, wherein, when the acrolein scavenger is dimercaprol, each thiol group in dimercaprol is linked to a linker, which is linked to a ligand.

29. The conjugate of claim 28, wherein each thiol group in dimercaprol is linked to a linker comprising a penicillamine and the linker is attached to the ligand by the penicillamine. wherein each thiol group in dimercaprol is linked to a linker comprising a penicillamine and the linker is attached to the ligand by the penicillamine.

30. The conjugate of claim 25, wherein the linker is hydrophilic or self-immolative.

31. (canceled)

32. The conjugate of claim 25, wherein the linker comprises a hydrophilic amino acid, a polyethylene glycol polymer, a peptidoglycan, a sugar, or a combination of two or more of the preceding.

33-37. (canceled)

38. A pharmaceutical composition comprising the conjugate of claim 25, and a pharmaceutically acceptable carrier.

39. The method of claim 23, wherein the patient has neurotrauma and delivering the payload to the to the patient constitutes treating the patient for neurotrauma.

40. The method of claim 39, wherein the neurotrauma is spinal trauma or brain trauma.

41. (canceled)
